SEQUENCE LISTING (134 sequences)

SEQ ID NO: 1 - DNA, 1335 bp, Triticum aestivum
  source 1..1335: mol_type = other DNA; note = TaCIPK14-4A
  CDS 1..1335: protein_id = 4; translation = MANRGKILMERYELGRLLGKGTFGKVHYARSLESNRSVAIKMLD KEKVLKVGLSEQIRREVTTMRLVAHKNIVQLHEVMATRNKIYFVMEYVKGGELFDKVA KSGKLTEGAAHKYFQQLISAVDYCHSQGVYHRDLKLENLLLDENENLKVSDFGLSALS ESKRQDGLLHTTCGTPAYVAPEVISKTGYDGAKSDIWSCGVILFVLVAGYLPFHGSNL MDMYRKIEQGDFRCPSWFSHKLQKLLFKILDPNPSTRASIQKIKESTWFRKGPRGTLA VKERTPSENVTTNAPPTAGVRPRKNTHEDVKPLMVTNLNAFEIISFSTGFDLSGLFIR EECRKETRFTSDKPASAIISKLEYVAKALNLRVRKKDNGVVKMQARKEGRNGAVQLDM EIFEITPSHHLIEMKQTSGDPLEYRELLEDIRPALKDIVWAWHGDDHHQQLE
atggcaaacagagggaagattctaatggagcggtacgagctgggaagattgttggggaaaggaacatttggcaaggtacactatgcaaggagcctagagtcgaaccgaagcgtcgccataaagatgctggacaaggagaaggtgctcaaggttgggctctcggagcaaatcaggcgtgaggtcacaaccatgcggttggtggcacacaagaacattgttcagcttcatgaggtcatggcgacacgaaacaaaatatactttgtcatggagtatgtgaaaggcggtgagctctttgacaaggttgcaaagagtggcaagctcacagagggtgctgcacataagtatttccagcagctcatcagtgcagtggattactgccacagccaaggcgtgtatcaccgggatctcaagctggagaacctgctcctggatgagaatgagaaccttaaggtctcggattttggattgagcgcactttcagagtcaaagaggcaagatggcttgctgcacaccacctgcggaacacccgcatatgtagctccggaggtcatcagcaagacaggttatgatggtgcgaaatcagatatctggtcttgtggtgttatcctttttgttcttgttgctggttatctccctttccatggttccaacttgatggacatgtaccggaagattgagcaaggagatttcaggtgccccagctggttctcacacaaactccagaagctcttgttcaagattctggaccccaatccaagcaccagggcatctatccagaagataaaagagtctacctggttccggaaaggtccaaggggaacccttgcagtgaaggagagaactcccagtgagaacgtcaccacaaatgctcctcctacagctggtgtgaggccaaggaagaacactcatgaagatgtgaagcccctgatggtgacaaacttaaatgcctttgagatcatctccttctccacggggtttgacctgtctggcctattcatccgagaggagtgcagaaaggagacaaggttcacttcagacaagcctgcttcagccatcatctcgaagctggaatatgttgcgaaagcgctgaatctcagggtaaggaagaaggacaatggcgtggtgaagatgcaagcgaggaaggaaggaaggaatggtgctgttcagttagacatggagatcttcgagatcacaccttcccaccacctcattgagatgaaacaaacaagtggtgatccactggagtaccgggagctattggaggacatccggccagcgctgaaggacatagtctgggcctggcacggagatgaccaccaccagcagctagagtag

SEQ ID NO: 2 - DNA, 1335 bp, Triticum aestivum
  source 1..1335: mol_type = other DNA; note = TaCIPK14-4B
  CDS 1..1335: protein_id = 5; translation = MANRGKILMERYELGRLLGKGTFGKVHYARSLESNQSVAIKMLD KEKVLKVGLSEQIRREVTTMRLVAHKNIVQLHEVMATRNKIYFVMEYVKGGELFDKVA KSGKLTEGAAHKYFQQLISAVDYCHSQGVYHRDLKLENLLLDENENLKVSDFGLSALS ESKRQDGLLHTTCGTPAYVAPEVISKTGYDGAKSDIWSCGVILFVLVAGYLPFHGSNL MDMYRKIEQGDFRCPSWFSHKLQKLLCKILDPNPSTRASIQKIKESTWFRKGPRGTLA VKERTPSENVTTNAPPTAGVRPRKNTHEDVQPLTVTNLNAFEIISFSTGFDLSGLFIQ EDCRKETRFTSDKPASAIISKLEYVAKALNLRVRKKDNGVVKMQARKEGRNGAVQLDM EIFEITPSHHLIEMKQTSGDPLEYRELLEDIRPALKDIVWAWHGDDHQQQLE
atggcaaacagagggaagattctaatggagcggtacgagctgggaagattgttggggaaaggaacattcggcaaggtgcactatgcaaggagcctagagtcgaaccaaagcgtcgccataaagatgctggacaaggagaaggtgctcaaggttgggctctcggagcaaatcaggcgtgaggtcacaaccatgcggctggtggcacacaagaacattgttcagcttcatgaggtcatggcgacacgaaacaaaatatactttgtcatggagtatgtgaaaggcggtgagctctttgacaaggttgcaaagagtggcaagctcacagagggtgctgcacataagtatttccagcagctcatcagtgcagtggattactgccacagccaaggcgtgtatcaccgggatctcaagctggagaacctgctcctggatgagaatgagaaccttaaggtctcagattttggactgagcgcactttcagagtcaaagaggcaagatggcttgctccacaccacctgcggaacacctgcatatgtagctccggaggtcatcagcaagacaggttacgatggtgcgaaatcagatatctggtcttgtggtgttatcctttttgttcttgttgctggttatctccctttccatggttccaacttgatggacatgtaccggaagattgaacaaggagatttcaggtgccccagctggttctcacacaaactccagaagctcttgtgcaagatcctggaccccaatccaagcaccagggcatctatccagaagataaaagagtctacctggtttcggaaaggtccaaggggcacccttgcagtgaaggagagaactcccagtgagaatgtcaccacaaatgctcctcctacagctggtgtgaggccaaggaagaacactcatgaagatgtgcagcccctgacggtgacaaacttaaatgcctttgagatcatctccttctccacggggtttgacctgtccggcctattcatccaagaggactgcagaaaggagacaaggttcacttcagacaagcctgcttcagccatcatctcgaagctggaatacgttgcaaaggcgctgaatctcagggtaaggaagaaggacaatggtgtggtgaagatgcaagcaaggaaggagggaaggaatggtgctgttcagttagacatggagatcttcgagatcacaccttcccaccacctcattgagatgaaacaaacaagtggtgatccgctggagtaccgggagctattggaggacatccggccagcgctgaaggacatagtctgggcctggcacggagatgaccaccagcagcagctagagtag

SEQ ID NO: 3 - DNA, 1335 bp, Triticum aestivum
  source 1..1335: mol_type = other DNA; note = TaCIPK14-4D
  CDS 1..1335: protein_id = 6; translation = MANRGKILMERYELGRLLGKGTFGKVHYARSLESNRSVAIKMLD KEKVLKVGLSEQIRREVTTMRLVAHKNIVQLHEVMATRNKIYFVMEYVKGGELFDKVA KSGKLTEGAAHKYFQQLISAVDYCHSQGVYHRDLKLENLLLDENENLKVSDFGLSALS ESKRQDGLLHTTCGTPAYVAPEVISKTGYDGAKSDIWSCGVILFVLVAGYLPFHGSNL MDMYRKIEQGDFRCPGWFSHKLQKLLLKILDPNPSTRASIQKIKESTWFRKGPRGTLA VKERTPSENVITNAPPTAGVRPRKNTHEDVKPLMVTNLNAFEIISFSTGFDLSGLFIQ EDCRKETRFTSDKPASTIISKLEYVAKALNLRVRKKDNGVVKMQARKEGRNGAVQLDM EIFEITPSHHLIEMKQTSGDPLEYRELLEDIRPALKDIVWAWHGDDHQQQLE
atggcaaacagagggaagattctaatggagcggtacgagctgggaagattgttggggaaaggaacattcggcaaggtgcactatgcaaggagcctagagtcgaaccgaagcgtcgccataaagatgctggacaaggagaaggtgctcaaggttgggctctcggagcaaatcaggcgtgaggtcacaaccatgcggttggtggcacacaagaacattgttcagcttcatgaggtcatggcgacacgaaacaaaatatactttgtcatggagtatgtgaaaggcggtgagctctttgacaaggttgcaaagagtggcaagctcacagagggtgctgcacataagtatttccagcagctcatcagtgcagtggattactgccacagccaaggcgtgtatcaccgggatctcaagctggagaacctgctcctggatgagaatgagaaccttaaggtctcggattttggattgagcgccctttcagagtcaaagaggcaagatggcttgctccacaccacctgcggaacacccgcatatgtagctccggaggtcatcagcaagacaggttacgatggtgcaaaatcagatatctggtcttgtggtgttatcctttttgttcttgttgctggttatctccctttccatggttccaacttgatggacatgtaccggaagattgagcaaggagatttcaggtgccccggctggttctcacacaaactccagaagctcttgctcaagatcctggaccccaatccaagcaccagggcatctatccagaagataaaagagtctacctggttccggaaaggtccaaggggcacccttgcagtgaaggagagaactcccagtgagaatgtcatcacaaatgctcctcctacagctggtgtgaggccaaggaagaacactcatgaagatgtgaagcccctaatggtgacaaacttaaatgcctttgagatcatctccttctccacggggtttgacctgtccggcctattcatccaagaggactgcagaaaggagacaaggttcacttcagacaagcctgcttcaaccatcatctcgaagctggaatatgttgcgaaggcgctgaatctcagggtaaggaagaaggacaatggcgtggtgaagatgcaagcgaggaaggagggaaggaatggtgctgtacagttagacatggagatcttcgagatcacaccttcccaccacctcattgagatgaaacaaacaagtggtgatccgctggagtaccgggagctattggaggacatccggccagcgctgaaggacatagtctgggcctggcacggagatgaccaccagcagcagctagagtag

SEQ ID NO: 4 - protein, 444 aa, Triticum aestivum
  source 1..444: note = Protein encoded by TaCIPK14-4A
MANRGKILMERYELGRLLGKGTFGKVHYARSLESNRSVAIKMLDKEKVLKVGLSEQIRREVTTMRLVAHKNIVQLHEVMATRNKIYFVMEYVKGGELFDKVAKSGKLTEGAAHKYFQQLISAVDYCHSQGVYHRDLKLENLLLDENENLKVSDFGLSALSESKRQDGLLHTTCGTPAYVAPEVISKTGYDGAKSDIWSCGVILFVLVAGYLPFHGSNLMDMYRKIEQGDFRCPSWFSHKLQKLLFKILDPNPSTRASIQKIKESTWFRKGPRGTLAVKERTPSENVTTNAPPTAGVRPRKNTHEDVKPLMVTNLNAFEIISFSTGFDLSGLFIREECRKETRFTSDKPASAIISKLEYVAKALNLRVRKKDNGVVKMQARKEGRNGAVQLDMEIFEITPSHHLIEMKQTSGDPLEYRELLEDIRPALKDIVWAWHGDDHHQQLE

SEQ ID NO: 5 - protein, 444 aa, Triticum aestivum
  source 1..444: note = Protein encoded by TaCIPK14-4B
MANRGKILMERYELGRLLGKGTFGKVHYARSLESNQSVAIKMLDKEKVLKVGLSEQIRREVTTMRLVAHKNIVQLHEVMATRNKIYFVMEYVKGGELFDKVAKSGKLTEGAAHKYFQQLISAVDYCHSQGVYHRDLKLENLLLDENENLKVSDFGLSALSESKRQDGLLHTTCGTPAYVAPEVISKTGYDGAKSDIWSCGVILFVLVAGYLPFHGSNLMDMYRKIEQGDFRCPSWFSHKLQKLLCKILDPNPSTRASIQKIKESTWFRKGPRGTLAVKERTPSENVTTNAPPTAGVRPRKNTHEDVQPLTVTNLNAFEIISFSTGFDLSGLFIQEDCRKETRFTSDKPASAIISKLEYVAKALNLRVRKKDNGVVKMQARKEGRNGAVQLDMEIFEITPSHHLIEMKQTSGDPLEYRELLEDIRPALKDIVWAWHGDDHQQQLE

SEQ ID NO: 6 - protein, 444 aa, Triticum aestivum
  source 1..444: note = Protein encoded by TaCIPK14-4D
MANRGKILMERYELGRLLGKGTFGKVHYARSLESNRSVAIKMLDKEKVLKVGLSEQIRREVTTMRLVAHKNIVQLHEVMATRNKIYFVMEYVKGGELFDKVAKSGKLTEGAAHKYFQQLISAVDYCHSQGVYHRDLKLENLLLDENENLKVSDFGLSALSESKRQDGLLHTTCGTPAYVAPEVISKTGYDGAKSDIWSCGVILFVLVAGYLPFHGSNLMDMYRKIEQGDFRCPGWFSHKLQKLLLKILDPNPSTRASIQKIKESTWFRKGPRGTLAVKERTPSENVITNAPPTAGVRPRKNTHEDVKPLMVTNLNAFEIISFSTGFDLSGLFIQEDCRKETRFTSDKPASTIISKLEYVAKALNLRVRKKDNGVVKMQARKEGRNGAVQLDMEIFEITPSHHLIEMKQTSGDPLEYRELLEDIRPALKDIVWAWHGDDHQQQLE

SEQ ID NO: 7 - RNA, 20 nt, synthetic construct
  source 1..20: mol_type = other RNA; note = gRNA1
ggcgtgaggtcacaaccatg

SEQ ID NO: 8 - RNA, 20 nt, synthetic construct
  source 1..20: mol_type = other RNA; note = gRNA2
gtcatggagtatgtgaaagg

SEQ ID NO: 9 - DNA, 24 bp, synthetic construct
  source 1..24: mol_type = other DNA; note = Primer VK005-6-R
gatgaagtggacggaaggaaggag

SEQ ID NO: 10 - DNA, 23 bp, synthetic construct
  source 1..23: mol_type = other DNA; note = Primer TaCIPK14-A-F
gattgttggggaaaggaacattt

SEQ ID NO: 11 - DNA, 24 bp, synthetic construct
  source 1..24: mol_type = other DNA; note = Primer TaCIPK14-A-R
ttgtctcctttctgcactcctctc

SEQ ID NO: 12 - DNA, 24 bp, synthetic construct
  source 1..24: mol_type = other DNA; note = Primer TaCIPK14-B-F
cgcagtccatttgaattatagcca

SEQ ID NO: 13 - DNA, 19 bp, synthetic construct
  source 1..19: mol_type = other DNA; note = Primer TaCIPK14-B-R
attcagcgcctttgcaacg

SEQ ID NO: 14 - DNA, 26 bp, synthetic construct
  source 1..26: mol_type = other DNA; note = Primer TaCIPK14-D-F
aagacatgagagtgttgttcactgtg

SEQ ID NO: 15 - DNA, 23 bp, synthetic construct
  source 1..23: mol_type = other DNA; note = Primer TaCIPK14-D-R
agctgtaggaggagcatttgtga

SEQ ID NO: 16 - DNA, 20 bp, synthetic construct
  source 1..20: mol_type = other DNA; note = Primer g1off1-F
cggagttcgggacctcggtg

SEQ ID NO: 17 - DNA, 22 bp, synthetic construct
  source 1..22: mol_type = other DNA; note = Primer g1off1-R
tcgccttcctcctcctcctgcc

SEQ ID NO: 18 - DNA, 25 bp, synthetic construct
  source 1..25: mol_type = other DNA; note = Primer g1off2-F
acctcctcctccttcctgtcgtctt

SEQ ID NO: 19 - DNA, 20 bp, synthetic construct
  source 1..20: mol_type = other DNA; note = Primer g1off2-R
tacggggttgctggcacttg

SEQ ID NO: 20 - DNA, 19 bp, synthetic construct
  source 1..19: mol_type = other DNA; note = Primer g1off3-F
cacggagacgcccagagac

SEQ ID NO: 21 - DNA, 19 bp, synthetic construct
  source 1..19: mol_type = other DNA; note = Primer g1off3-R
cgcacggtaggagcaacgg

SEQ ID NO: 22 - DNA, 25 bp, synthetic construct
  source 1..25: mol_type = other DNA; note = Primer g1off4-F
tatttcctccatcttgctctcttcc

SEQ ID NO: 23 - DNA, 26 bp, synthetic construct
  source 1..26: mol_type = other DNA; note = Primer g1off-4-R
caagtgtctactctctccatccacaa

SEQ ID NO: 24 - DNA, 20 bp, synthetic construct
  source 1..20: mol_type = other DNA; note = Primer g1off5-F
cgtgtgcctgctttttgctg

SEQ ID NO: 25 - DNA, 23 bp, synthetic construct
  source 1..23: mol_type = other DNA; note = Primer g1off5-R
cgccagtgttgatgtgagattgt

SEQ ID NO: 26 - DNA, 19 bp, synthetic construct
  source 1..19: mol_type = other DNA; note = Primer g1off6-F
ggggaggatgtgggcagat

SEQ ID NO: 27 - DNA, 22 bp, synthetic construct
  source 1..22: mol_type = other DNA; note = Primer g1off6-R
cgcaacgaggaccaacacttta

SEQ ID NO: 28 - DNA, 27 bp, synthetic construct
  source 1..27: mol_type = other DNA; note = Primer g1off7-F
ctcatccatttttatcacaaccttaga

SEQ ID NO: 29 - DNA, 19 bp, synthetic construct
  source 1..19: mol_type = other DNA; note = Primer g1off7-R
gctgaccgccagacccact

SEQ ID NO: 30 - DNA, 23 bp, Triticum aestivum
  source 1..23: mol_type = other DNA; note = OFF1
agctcgaggtcacaaccatgggg

SEQ ID NO: 31 - DNA, 23 bp, Triticum aestivum
  source 1..23: mol_type = other DNA; note = OFF2
agctagaggtcacaaccatgggg

SEQ ID NO: 32 - DNA, 23 bp, Triticum aestivum
  source 1..23: mol_type = other DNA; note = OFF3
agctcgaggtcacaaccatgggg

SEQ ID NO: 33 - DNA, 23 bp, Triticum aestivum
  source 1..23: mol_type = other DNA; note = OFF4
cgagtgaggtcgcaaccatgcgg

SEQ ID NO: 34 - DNA, 23 bp, Triticum aestivum
  source 1..23: mol_type = other DNA; note = OFF5
ggttctgtccttctctgcaatgg

SEQ ID NO: 35 - DNA, 23 bp, Triticum aestivum
  source 1..23: mol_type = other DNA; note = OFF6
aattttctccttcgctgcaaagg

SEQ ID NO: 36 - DNA, 23 bp, Triticum aestivum
  source 1..23: mol_type = other DNA; note = OFF7
tgctctctacttcactgcaatgg

SEQ ID NO: 37 - DNA, 29 bp, Triticum aestivum
  source 1..29: mol_type = other DNA; note = Fragment of TaCIPK14-4A of Fielder
gtcatggagtatgtgaaaggcggtgagct

SEQ ID NO: 38 - DNA, 30 bp, Triticum aestivum
  source 1..30: mol_type = other DNA; note = Fragment of TaCIPK14-4A of T0-4
gtcatggagtatgtgaaaaggcggtgagct

SEQ ID NO: 39 - DNA, 29 bp, Triticum aestivum
  source 1..29: mol_type = other DNA; note = Fragment of TaCIPK14-4A of T0-12
gtcatggagtatgtgaaaggcggtgagct

SEQ ID NO: 40 - DNA, 29 bp, Triticum aestivum
  source 1..29: mol_type = other DNA; note = Fragment of TaCIPK14-4B of Fielder
gtcatggagtatgtgaaaggcggtgagct

SEQ ID NO: 41 - DNA, 30 bp, Triticum aestivum; identical to SEQ ID NO: 38
  source 1..30: mol_type = other DNA; note = Fragment of TaCIPK14-4B of T0-4

SEQ ID NO: 42 - DNA, 29 bp, Triticum aestivum
  source 1..29: mol_type = other DNA; note = Fragment of TaCIPK14-4B of T0-12
gtcatggagtatgggagaggagctcattt

SEQ ID NO: 43 - DNA, 29 bp, Triticum aestivum
  source 1..29: mol_type = other DNA; note = Fragment of TaCIPK14-4D of Fielder
gtcatggagtatgtgaaaggcggtgagct

SEQ ID NO: 44 - DNA, 29 bp, Triticum aestivum
  source 1..29: mol_type = other DNA; note = Fragment of TaCIPK14-4D of T0-4
gtcatggagtatgtgaaaggcggtgagct

SEQ ID NO: 45 - DNA, 29 bp, Triticum aestivum
  source 1..29: mol_type = other DNA; note = Fragment of TaCIPK14-4D of T0-12
gtcatggagtatgtgaggggagctctttg

SEQ ID NO: 46 - DNA, 29 bp, Triticum aestivum
  source 1..29: mol_type = other DNA; note = Fragment of TaCIPK14-4A of Fielder
ggcgtgaggtcacaaccatgcggttggtg

SEQ ID NO: 47 - DNA, 29 bp, Triticum aestivum
  source 1..29: mol_type = other DNA; note = Fragment of TaCIPK14-4A of T0-21
ggcgtgaggtcacaaccatgcggttggtg

SEQ ID NO: 48 - DNA, 29 bp, Triticum aestivum
  source 1..29: mol_type = other DNA; note = Fragment of TaCIPK14-4B of Fielder
ggcgtgaggtcacaaccatgcggttggtg

SEQ ID NO: 49 - DNA, 29 bp, Triticum aestivum
  source 1..29: mol_type = other DNA; note = Fragment of TaCIPK14-4B of T0-21
ggcgtgaggtcacaaccatgcggtgggct

SEQ ID NO: 50 - DNA, 29 bp, Triticum aestivum
  source 1..29: mol_type = other DNA; note = Fragment of TaCIPK14-4D of Fielder
ggcgtgaggtcacaaccatgcggttggtg

SEQ ID NO: 51 - DNA, 29 bp, Triticum aestivum
  source 1..29: mol_type = other DNA; note = Fragment of TaCIPK14-4D of T0-21
ggcgtgaggtcgttggtggcacacaagaa

SEQ ID NO: 52 - DNA, 33 bp, Triticum aestivum
  source 1..33: mol_type = other DNA; note = Fragment of TaCIPK14-4A of Fielder
caggcgtgaggtcacaaccatgcggttggtggc

SEQ ID NO: 53 - DNA, 34 bp, Triticum aestivum
  source 1..34: mol_type = other DNA; note = Fragment of TaCIPK14-4A of Fielder
tatactttgtcatggagtatgtgaaaggcggtga

SEQ ID NO: 54 - DNA, 33 bp, Triticum aestivum
  source 1..33: mol_type = other DNA; note = Fragment of TaCIPK14-4B of Fielder
caggcgtgaggtcacaaccatgcggctggtggc

SEQ ID NO: 55 - DNA, 34 bp, Triticum aestivum; identical to SEQ ID NO: 53
  source 1..34: mol_type = other DNA; note = Fragment of TaCIPK14-4B of Fielder

SEQ ID NO: 56 - DNA, 33 bp, Triticum aestivum; identical to SEQ ID NO: 52
  source 1..33: mol_type = other DNA; note = Fragment of TaCIPK14-4D of Fielder

SEQ ID NO: 57 - DNA, 33 bp, Triticum aestivum
  source 1..33: mol_type = other DNA; note = Fragment of TaCIPK14-4D of Fielder
tatactttgtcatggagtatggaaaggcggtga

SEQ ID NO: 58 - DNA, 33 bp, Triticum aestivum; identical to SEQ ID NO: 52
  source 1..33: mol_type = other DNA; note = Fragment of TaCIPK14-4A of KO-1

SEQ ID NO: 59 - DNA, 35 bp, Triticum aestivum
  source 1..35: mol_type = other DNA; note = Fragment of TaCIPK14-4A of KO-1
tatactttgtcatggagtatgtgaaaaggcggtga

SEQ ID NO: 60 - DNA, 32 bp, Triticum aestivum
  source 1..32: mol_type = other DNA; note = Fragment of TaCIPK14-4B of KO-1
caggcgtgaggtcacaaccatgcggctggtgg

SEQ ID NO: 61 - DNA, 29 bp, Triticum aestivum
  source 1..29: mol_type = other DNA; note = Fragment of TaCIPK14-4B of KO-1
tatactttgtcatggagtaaaggcggtga

SEQ ID NO: 62 - DNA, 33 bp, Triticum aestivum; identical to SEQ ID NO: 52
  source 1..33: mol_type = other DNA; note = Fragment of TaCIPK14-4D of KO-1

SEQ ID NO: 63 - DNA, 29 bp, Triticum aestivum
  source 1..29: mol_type = other DNA; note = Fragment of TaCIPK14-4D of KO-1
tatactttgtcatggagtataggcggtga

SEQ ID NO: 64 - DNA, 33 bp, Triticum aestivum; identical to SEQ ID NO: 52
  source 1..33: mol_type = other DNA; note = Fragment of TaCIPK14-4A of KO-2

SEQ ID NO: 65 - DNA, 35 bp, Triticum aestivum; identical to SEQ ID NO: 59
  source 1..35: mol_type = other DNA; note = Fragment of TaCIPK14-4A of KO-2

SEQ ID NO: 66 - DNA, 33 bp, Triticum aestivum; identical to SEQ ID NO: 54
  source 1..33: mol_type = other DNA; note = Fragment of TaCIPK14-4B of KO-2

SEQ ID NO: 67 - DNA, 35 bp, Triticum aestivum; identical to SEQ ID NO: 59
  source 1..35: mol_type = other DNA; note = Fragment of TaCIPK14-4B of KO-2

SEQ ID NO: 68 - DNA, 33 bp, Triticum aestivum; identical to SEQ ID NO: 52
  source 1..33: mol_type = other DNA; note = Fragment of TaCIPK14-4D of KO-2

SEQ ID NO: 69 - DNA, 28 bp, Triticum aestivum
  source 1..28: mol_type = other DNA; note = Fragment of TaCIPK14-4D of KO-2
tatacttgtcatggagtataggcggtga

SEQ ID NO: 70 - DNA, 33 bp, Triticum aestivum; identical to SEQ ID NO: 52
  source 1..33: mol_type = other DNA; note = Fragment of TaCIPK14-4A of KO-3

SEQ ID NO: 71 - DNA, 35 bp, Triticum aestivum; identical to SEQ ID NO: 59
  source 1..35: mol_type = other DNA; note = Fragment of TaCIPK14-4A of KO-3

SEQ ID NO: 72 - DNA, 33 bp, Triticum aestivum; identical to SEQ ID NO: 54
  source 1..33: mol_type = other DNA; note = Fragment of TaCIPK14-4B of KO-3

SEQ ID NO: 73 - DNA, 29 bp, Triticum aestivum
  source 1..29: mol_type = other DNA; note = Fragment of TaCIPK14-4B of KO-3
tatactttgtcatggagtaaaggcggtga

SEQ ID NO: 74 - DNA, 33 bp, Triticum aestivum; identical to SEQ ID NO: 52
  source 1..33: mol_type = other DNA; note = Fragment of TaCIPK14-4D of KO-3

SEQ ID NO: 75 - DNA, 33 bp, Triticum aestivum
  source 1..33: mol_type = other DNA; note = Fragment of TaCIPK14-4D of KO-3
tatactttgtcatggagtatgtgaaggcggtga

SEQ ID NO: 76 - DNA, 33 bp, Triticum aestivum; identical to SEQ ID NO: 52
  source 1..33: mol_type = other DNA; note = Fragment of TaCIPK14-4A of KO-4

SEQ ID NO: 77 - DNA, 35 bp, Triticum aestivum; identical to SEQ ID NO: 59
  source 1..35: mol_type = other DNA; note = Fragment of TaCIPK14-4A of KO-4

SEQ ID NO: 78 - DNA, 19 bp, Triticum aestivum
  source 1..19: mol_type = other DNA; note = Fragment of TaCIPK14-4B of KO-4
caggcgtgaggtcacaacc

SEQ ID NO: 79 - DNA, 22 bp, Triticum aestivum
  source 1..22: mol_type = other DNA; note = Fragment of TaCIPK14-4D of KO-4
caggcgtgaggtcgttggtggc

SEQ ID NO: 80 - DNA, 34 bp, Triticum aestivum; identical to SEQ ID NO: 53
  source 1..34: mol_type = other DNA; note = Fragment of TaCIPK14-4D of KO-4

SEQ ID NO: 81 - DNA, 33 bp, Triticum aestivum; identical to SEQ ID NO: 52
  source 1..33: mol_type = other DNA; note = Fragment of TaCIPK14-4A of KO-5

SEQ ID NO: 82 - DNA, 35 bp, Triticum aestivum; identical to SEQ ID NO: 59
  source 1..35: mol_type = other DNA; note = Fragment of TaCIPK14-4A of KO-5

SEQ ID NO: 83 - DNA, 33 bp, Triticum aestivum; identical to SEQ ID NO: 54
  source 1..33: mol_type = other DNA; note = Fragment of TaCIPK14-4B of KO-5

SEQ ID NO: 84 - DNA, 35 bp, Triticum aestivum; identical to SEQ ID NO: 59
  source 1..35: mol_type = other DNA; note = Fragment of TaCIPK14-4B of KO-5

SEQ ID NO: 85 - DNA, 21 bp, Triticum aestivum
  source 1..21: mol_type = other DNA; note = Fragment of TaCIPK14-4D of KO-5
caggcgtgaggtcgttggggc

SEQ ID NO: 86 - DNA, 34 bp, Triticum aestivum; identical to SEQ ID NO: 53
  source 1..34: mol_type = other DNA; note = Fragment of TaCIPK14-4D of KO-5

SEQ ID NO: 87 - DNA, 1336 bp, Triticum aestivum
  source 1..1336: mol_type = other DNA; note = TaCIPK14-4A of KO-1
atggcaaacagagggaagattctaatggagcggtacgagctgggaagattgttggggaaaggaacatttggcaaggtacactatgcaaggagcctagagtcgaaccgaagcgtcgccataaagatgctggacaaggagaaggtgctcaaggttgggctctcggagcaaatcaggcgtgaggtcacaaccatgcggttggtggcacacaagaacattgttcagcttcatgaggtcatggcgacacgaaacaaaatatactttgtcatggagtatgtgaaaaggcggtgagctctttgacaaggttgcaaagagtggcaagctcacagagggtgctgcacataagtatttccagcagctcatcagtgcagtggattactgccacagccaaggcgtgtatcaccgggatctcaagctggagaacctgctcctggatgagaatgagaaccttaaggtctcggattttggattgagcgcactttcagagtcaaagaggcaagatggcttgctgcacaccacctgcggaacacccgcatatgtagctccggaggtcatcagcaagacaggttatgatggtgcgaaatcagatatctggtcttgtggtgttatcctttttgttcttgttgctggttatctccctttccatggttccaacttgatggacatgtaccggaagattgagcaaggagatttcaggtgccccagctggttctcacacaaactccagaagctcttgttcaagattctggaccccaatccaagcaccagggcatctatccagaagataaaagagtctacctggttccggaaaggtccaaggggaacccttgcagtgaaggagagaactcccagtgagaacgtcaccacaaatgctcctcctacagctggtgtgaggccaaggaagaacactcatgaagatgtgaagcccctgatggtgacaaacttaaatgcctttgagatcatctccttctccacggggtttgacctgtctggcctattcatccgagaggagtgcagaaaggagacaaggttcacttcagacaagcctgcttcagccatcatctcgaagctggaatatgttgcgaaagcgctgaatctcagggtaaggaagaaggacaatggcgtggtgaagatgcaagcgaggaaggaaggaaggaatggtgctgttcagttagacatggagatcttcgagatcacaccttcccaccacctcattgagatgaaacaaacaagtggtgatccactggagtaccgggagctattggaggacatccggccagcgctgaaggacatagtctgggcctggcacggagatgaccaccaccagcagctagagtag

SEQ ID NO: 88 - DNA, 1330 bp, Triticum aestivum
  source 1..1330: mol_type = other DNA; note = TaCIPK14-4B of KO-1
atggcaaacagagggaagattctaatggagcggtacgagctgggaagattgttggggaaaggaacattcggcaaggtgcactatgcaaggagcctagagtcgaaccaaagcgtcgccataaagatgctggacaaggagaaggtgctcaaggttgggctctcggagcaaatcaggcgtgaggtcacaaccatgcggctggtggcacacaagaacattgttcagcttcatgaggtcatggcgacacgaaacaaaatatactttgtcatggagtaaaggcggtgagctctttgacaaggttgcaaagagtggcaagctcacagagggtgctgcacataagtatttccagcagctcatcagtgcagtggattactgccacagccaaggcgtgtatcaccgggatctcaagctggagaacctgctcctggatgagaatgagaaccttaaggtctcagattttggactgagcgcactttcagagtcaaagaggcaagatggcttgctccacaccacctgcggaacacctgcatatgtagctccggaggtcatcagcaagacaggttacgatggtgcgaaatcagatatctggtcttgtggtgttatcctttttgttcttgttgctggttatctccctttccatggttccaacttgatggacatgtaccggaagattgaacaaggagatttcaggtgccccagctggttctcacacaaactccagaagctcttgtgcaagatcctggaccccaatccaagcaccagggcatctatccagaagataaaagagtctacctggtttcggaaaggtccaaggggcacccttgcagtgaaggagagaactcccagtgagaatgtcaccacaaatgctcctcctacagctggtgtgaggccaaggaagaacactcatgaagatgtgcagcccctgacggtgacaaacttaaatgcctttgagatcatctccttctccacggggtttgacctgtccggcctattcatccaagaggactgcagaaaggagacaaggttcacttcagacaagcctgcttcagccatcatctcgaagctggaatacgttgcaaaggcgctgaatctcagggtaaggaagaaggacaatggtgtggtgaagatgcaagcaaggaaggagggaaggaatggtgctgttcagttagacatggagatcttcgagatcacaccttcccaccacctcattgagatgaaacaaacaagtggtgatccgctggagtaccgggagctattggaggacatccggccagcgctgaaggacatagtctgggcctggcacggagatgaccaccagcagcagctagagtag

SEQ ID NO: 89 - DNA, 1330 bp, Triticum aestivum
  source 1..1330: mol_type = other DNA; note = TaCIPK14-4D of KO-1
atggcaaacagagggaagattctaatggagcggtacgagctgggaagattgttggggaaaggaacattcggcaaggtgcactatgcaaggagcctagagtcgaaccgaagcgtcgccataaagatgctggacaaggagaaggtgctcaaggttgggctctcggagcaaatcaggcgtgaggtcacaaccatgcggttggtggcacacaagaacattgttcagcttcatgaggtcatggcgacacgaaacaaaatatactttgtcatggagtataggcggtgagctctttgacaaggttgcaaagagtggcaagctcacagagggtgctgcacataagtatttccagcagctcatcagtgcagtggattactgccacagccaaggcgtgtatcaccgggatctcaagctggagaacctgctcctggatgagaatgagaaccttaaggtctcggattttggattgagcgccctttcagagtcaaagaggcaagatggcttgctccacaccacctgcggaacacccgcatatgtagctccggaggtcatcagcaagacaggttacgatggtgcaaaatcagatatctggtcttgtggtgttatcctttttgttcttgttgctggttatctccctttccatggttccaacttgatggacatgtaccggaagattgagcaaggagatttcaggtgccccggctggttctcacacaaactccagaagctcttgctcaagatcctggaccccaatccaagcaccagggcatctatccagaagataaaagagtctacctggttccggaaaggtccaaggggcacccttgcagtgaaggagagaactcccagtgagaatgtcatcacaaatgctcctcctacagctggtgtgaggccaaggaagaacactcatgaagatgtgaagcccctaatggtgacaaacttaaatgcctttgagatcatctccttctccacggggtttgacctgtccggcctattcatccaagaggactgcagaaaggagacaaggttcacttcagacaagcctgcttcaaccatcatctcgaagctggaatatgttgcgaaggcgctgaatctcagggtaaggaagaaggacaatggcgtggtgaagatgcaagcgaggaaggagggaaggaatggtgctgtacagttagacatggagatcttcgagatcacaccttcccaccacctcattgagatgaaacaaacaagtggtgatccgctggagtaccgggagctattggaggacatccggccagcgctgaaggacatagtctgggcctggcacggagatgaccaccagcagcagctagagtag

SEQ ID NO: 90 - DNA, 1336 bp, Triticum aestivum; identical to SEQ ID NO: 87
  source 1..1336: mol_type = other DNA; note = TaCIPK14-4A of KO-2

SEQ ID NO: 91 - DNA, 1336 bp, Triticum aestivum; identical to SEQ ID NO: 87
  source 1..1336: mol_type = other DNA; note = TaCIPK14-4B of KO-2

SEQ ID NO: 92 - DNA, 1330 bp, Triticum aestivum; identical to SEQ ID NO: 89
  source 1..1330: mol_type = other DNA; note = TaCIPK14-4D of KO-2

SEQ ID NO: 93 - DNA, 1336 bp, Triticum aestivum; identical to SEQ ID NO: 87
  source 1..1336: mol_type = other DNA; note = TaCIPK14-4A of KO-3

SEQ ID NO: 94 - DNA, 1330 bp, Triticum aestivum; identical to SEQ ID NO: 88
  source 1..1330: mol_type = other DNA; note = TaCIPK14-4B of KO-3

SEQ ID NO: 95 - DNA, 1334 bp, Triticum aestivum
  source 1..1334: mol_type = other DNA; note = TaCIPK14-4D of KO-3
atggcaaacagagggaagattctaatggagcggtacgagctgggaagattgttggggaaaggaacattcggcaaggtgcactatgcaaggagcctagagtcgaaccgaagcgtcgccataaagatgctggacaaggagaaggtgctcaaggttgggctctcggagcaaatcaggcgtgaggtcacaaccatgcggttggtggcacacaagaacattgttcagcttcatgaggtcatggcgacacgaaacaaaatatactttgtcatggagtatgtgaaggcggtgagctctttgacaaggttgcaaagagtggcaagctcacagagggtgctgcacataagtatttccagcagctcatcagtgcagtggattactgccacagccaaggcgtgtatcaccgggatctcaagctggagaacctgctcctggatgagaatgagaaccttaaggtctcggattttggattgagcgccctttcagagtcaaagaggcaagatggcttgctccacaccacctgcggaacacccgcatatgtagctccggaggtcatcagcaagacaggttacgatggtgcaaaatcagatatctggtcttgtggtgttatcctttttgttcttgttgctggttatctccctttccatggttccaacttgatggacatgtaccggaagattgagcaaggagatttcaggtgccccggctggttctcacacaaactccagaagctcttgctcaagatcctggaccccaatccaagcaccagggcatctatccagaagataaaagagtctacctggttccggaaaggtccaaggggcacccttgcagtgaaggagagaactcccagtgagaatgtcatcacaaatgctcctcctacagctggtgtgaggccaaggaagaacactcatgaagatgtgaagcccctaatggtgacaaacttaaatgcctttgagatcatctccttctccacggggtttgacctgtccggcctattcatccaagaggactgcagaaaggagacaaggttcacttcagacaagcctgcttcaaccatcatctcgaagctggaatatgttgcgaaggcgctgaatctcagggtaaggaagaaggacaatggcgtggtgaagatgcaagcgaggaaggagggaaggaatggtgctgtacagttagacatggagatcttcgagatcacaccttcccaccacctcattgagatgaaacaaacaagtggtgatccgctggagtaccgggagctattggaggacatccggccagcgctgaaggacatagtctgggcctggcacggagatgaccaccagcagcagctagagtag

SEQ ID NO: 96 - DNA, 1336 bp, Triticum aestivum; identical to SEQ ID NO: 87
  source 1..1336: mol_type = other DNA; note = TaCIPK14-4A of KO-4

SEQ ID NO: 97 - DNA, 1246 bp, Triticum aestivum
  source 1..1246: mol_type = other DNA; note = TaCIPK14-4B of KO-4
atggcaaacagagggaagattctaatggagcggtacgagctgggaagattgttggggaaaggaacattcggcaaggtgcactatgcaaggagcctagagtcgaaccaaagcgtcgccataaagatgctggacaaggagaaggtgctcaaggttgggctctcggagcaaatcaggcgtgaggtcacaaccaggcggtgagctctttgacaaggttgcaaagagtggcaagctcacagagggtgctgcacataagtatttccagcagctcatcagtgcagtggattactgccacagccaaggcgtgtatcaccgggatctcaagctggagaacctgctcctggatgagaatgagaaccttaaggtctcagattttggactgagcgcactttcagagtcaaagaggcaagatggcttgctccacaccacctgcggaacacctgcatatgtagctccggaggtcatcagcaagacaggttacgatggtgcgaaatcagatatctggtcttgtggtgttatcctttttgttcttgttgctggttatctccctttccatggttccaacttgatggacatgtaccggaagattgaacaaggagatttcaggtgccccagctggttctcacacaaactccagaagctcttgtgcaagatcctggaccccaatccaagcaccagggcatctatccagaagataaaagagtctacctggtttcggaaaggtccaaggggcacccttgcagtgaaggagagaactcccagtgagaatgtcaccacaaatgctcctcctacagctggtgtgaggccaaggaagaacactcatgaagatgtgcagcccctgacggtgacaaacttaaatgcctttgagatcatctccttctccacggggtttgacctgtccggcctattcatccaagaggactgcagaaaggagacaaggttcacttcagacaagcctgcttcagccatcatctcgaagctggaatacgttgcaaaggcgctgaatctcagggtaaggaagaaggacaatggtgtggtgaagatgcaagcaaggaaggagggaaggaatggtgctgttcagttagacatggagatcttcgagatcacaccttcccaccacctcattgagatgaaacaaacaagtggtgatccgctggagtaccgggagctattggaggacatccggccagcgctgaaggacatagtctgggcctggcacggagatgaccaccagcagcagctagagtag

SEQ ID NO: 98 - DNA, 1324 bp, Triticum aestivum
  source 1..1324: mol_type = other DNA; note = TaCIPK14-4D of KO-4
atggcaaacagagggaagattctaatggagcggtacgagctgggaagattgttggggaaaggaacattcggcaaggtgcactatgcaaggagcctagagtcgaaccgaagcgtcgccataaagatgctggacaaggagaaggtgctcaaggttgggctctcggagcaaatcaggcgtgaggtcgttggtggcacacaagaacattgttcagcttcatgaggtcatggcgacacgaaacaaaatatactttgtcatggagtatgtgaaaggcggtgagctctttgacaaggttgcaaagagtggcaagctcacagagggtgctgcacataagtatttccagcagctcatcagtgcagtggattactgccacagccaaggcgtgtatcaccgggatctcaagctggagaacctgctcctggatgagaatgagaaccttaaggtctcggattttggattgagcgccctttcagagtcaaagaggcaagatggcttgctccacaccacctgcggaacacccgcatatgtagctccggaggtcatcagcaagacaggttacgatggtgcaaaatcagatatctggtcttgtggtgttatcctttttgttcttgttgctggttatctccctttccatggttccaacttgatggacatgtaccggaagattgagcaaggagatttcaggtgccccggctggttctcacacaaactccagaagctcttgctcaagatcctggaccccaatccaagcaccagggcatctatccagaagataaaagagtctacctggttccggaaaggtccaaggggcacccttgcagtgaaggagagaactcccagtgagaatgtcatcacaaatgctcctcctacagctggtgtgaggccaaggaagaacactcatgaagatgtgaagcccctaatggtgacaaacttaaatgcctttgagatcatctccttctccacggggtttgacctgtccggcctattcatccaagaggactgcagaaaggagacaaggttcacttcagacaagcctgcttcaaccatcatctcgaagctggaatatgttgcgaaggcgctgaatctcagggtaaggaagaaggacaatggcgtggtgaagatgcaagcgaggaaggagggaaggaatggtgctgtacagttagacatggagatcttcgagatcacaccttcccaccacctcattgagatgaaacaaacaagtggtgatccgctggagtaccgggagctattggaggacatccggccagcgctgaaggacatagtctgggcctggcacggagatgaccaccagcagcagctagagtag

SEQ ID NO: 99 - DNA, 1336 bp, Triticum aestivum; identical to SEQ ID NO: 87
  source 1..1336: mol_type = other DNA; note = TaCIPK14-4A of KO-5

SEQ ID NO: 100 - DNA, 1336 bp, Triticum aestivum; identical to SEQ ID NO: 87
  source 1..1336: mol_type = other DNA; note = TaCIPK14-4B of KO-5

SEQ ID NO: 101 - DNA, 1324 bp, Triticum aestivum; identical to SEQ ID NO: 98
  source 1..1324: mol_type = other DNA; note = TaCIPK14-4D of KO-5

SEQ ID NO: 102 - protein, 105 aa, synthetic construct
  source 1..105: note = Deduced amino acid sequence fragment of TaCIPK14-4A of Fielder
IRREVTTMRLVAHKNIVQLHEVMATRNKIYFVMEYVKGGELFDKVAKSGKLTEGAAHKYFQQLISAVDYCHSQGVYHRDLKLENLLLDENENLKVSDFGLSALSE

SEQ ID NO: 103 - protein, 106 aa, synthetic construct
  source 1..106: note = Deduced amino acid sequence fragment of TaCIPK14-4B of Fielder
IRREVTTMRLVAHKNIVQLHEVMATRTNKIYFVMEYVKGGELFDKVAKSGKLTEGAAHKYFQQLISAVDYCHSQGVYHRDLKLENLLLDENENLKVSDFGLSALSE

SEQ ID NO: 104 - protein, 105 aa, synthetic construct; identical to SEQ ID NO: 102
  source 1..105: note = Deduced amino acid sequence fragment of TaCIPK14-4D of Fielder

SEQ ID NO: 105 - protein, 39 aa, synthetic construct
  source 1..39: note = Deduced amino acid sequence fragment of TaCIPK14-4A of KO-1
IRREVTTMRLVAHKNIVQLHEVMATRNKIYFVMEYVKRR

SEQ ID NO: 106 - protein, 34 aa, synthetic construct
  source 1..34: note = Deduced amino acid sequence fragment of TaCIPK14-4B of KO-1
IRREVTTMRLVAHKNIVQLHEVMATRNKIYFVME

SEQ ID NO: 107 - protein, 93 aa, synthetic construct
  source 1..93: note = Deduced amino acid sequence fragment of TaCIPK14-4D of KO-1
MANRGKILMERYELGRLLGKGTFGKVHYARSLESNRSVAIKMLDKEKVLKVGLSEQIRREVTTMRLVAHKNIVQLHEVMATRNKIYFVMEYRR

SEQ ID NO: 108 - protein, 39 aa, synthetic construct; identical to SEQ ID NO: 105
  source 1..39: note = Deduced amino acid sequence fragment of TaCIPK14-4A of KO-2

SEQ ID NO: 109 - protein, 39 aa, synthetic construct; identical to SEQ ID NO: 105
  source 1..39: note = Deduced amino acid sequence fragment of TaCIPK14-4B of KO-2

SEQ ID NO: 110 - protein, 37 aa, synthetic construct
  source 1..37: note = Deduced amino acid sequence fragment of TaCIPK14-4D of KO-2
IRREVTTMRLVAHKNIVQLHEVMATRNKIYFVMEYRR

SEQ ID NO: 111 - protein, 39 aa, synthetic construct; identical to SEQ ID NO: 105
  source 1..39: note = Deduced amino acid sequence fragment of TaCIPK14-4A of KO-3

SEQ ID NO: 112 - protein, 34 aa, synthetic construct; identical to SEQ ID NO: 106
  source 1..34: note = Deduced amino acid sequence fragment of TaCIPK14-4B of KO-3

SEQ ID NO: 113 - protein, 99 aa, synthetic construct
  source 1..99: note = Deduced amino acid sequence fragment of TaCIPK14-4D of KO-3
IRREVTTMRLVAHKNIVQLHEVMATRNKIYFVMEYVKAVSSLTRLQRVASSQRVLHISISSSSSVQWITATAKACITGISSWRTCSWMRMRTLRSRILD

SEQ ID NO: 114 - protein, 39 aa, synthetic construct; identical to SEQ ID NO: 105
  source 1..39: note = Deduced amino acid sequence fragment of TaCIPK14-4A of KO-4

SEQ ID NO: 115 - protein, 9 aa, synthetic construct
  source 1..9: note = Deduced amino acid sequence fragment of TaCIPK14-4B of KO-4
IRREVTTRR

SEQ ID NO: 116 - protein, 16 aa, synthetic construct
  source 1..16: note = Deduced amino acid sequence fragment of TaCIPK14-4D of KO-4
IRREVVGGTQEHCSAS

SEQ ID NO: 117 - protein, 39 aa, synthetic construct; identical to SEQ ID NO: 105
  source 1..39: note = Deduced amino acid sequence fragment of TaCIPK14-4A of KO-5

SEQ ID NO: 118 - protein, 39 aa, synthetic construct; identical to SEQ ID NO: 105
  source 1..39: note = Deduced amino acid sequence fragment of TaCIPK14-4B of KO-5

SEQ ID NO: 119 - protein, 16 aa, synthetic construct
  source 1..16: note = Deduced amino acid sequence fragment of TaCIPK14-4D of KO-5
IRREVVGGTQEHCSAS

SEQ ID NO: 120 - protein, 95 aa, synthetic construct
  source 1..95: note = Deduced amino acid sequence TaCIPK14-4A of KO-1
MANRGKILMERYELGRLLGKGTFGKVHYARSLESNRSVAIKMLDKEKVLKVGLSEQIRREVTTMRLVAHKNIVQLHEVMATRNKIYFVMEYVKRR

SEQ ID NO: 121 - protein, 90 aa, synthetic construct
  source 1..90: note = Deduced amino acid sequence TaCIPK14-4B of KO-1
MANRGKILMERYELGRLLGKGTFGKVHYARSLESNQSVAIKMLDKEKVLKVGLSEQIRREVTTMRLVAHKNIVQLHEVMATRNKIYFVME

SEQ ID NO: 122 - protein, 93 aa, synthetic construct; identical to SEQ ID NO: 107
  source 1..93: note = Deduced amino acid sequence TaCIPK14-4D of KO-1

SEQ ID NO: 123 - protein, 95 aa, synthetic construct; identical to SEQ ID NO: 120
  source 1..95: note = Deduced amino acid sequence TaCIPK14-4A of KO-2

SEQ ID NO: 124 - protein, 95 aa, synthetic construct
  source 1..95: note = Deduced amino acid sequence TaCIPK14-4B of KO-2
MANRGKILMERYELGRLLGKGTFGKVHYARSLESNQSVAIKMLDKEKVLKVGLSEQIRREVTTMRLVAHKNIVQLHEVMATRNKIYFVMEYVKRR

SEQ ID NO: 125 - protein, 93 aa, synthetic construct; identical to SEQ ID NO: 107
  source 1..93: note = Deduced amino acid sequence TaCIPK14-4D of KO-2

SEQ ID NO: 126 - protein, 95 aa, synthetic construct; identical to SEQ ID NO: 120
  source 1..95: note = Deduced amino acid sequence TaCIPK14-4A of KO-3

SEQ ID NO: 127 - protein, 90 aa, synthetic construct; identical to SEQ ID NO: 121
  source 1..90: note = Deduced amino acid sequence TaCIPK14-4B of KO-3

SEQ ID NO: 128 - protein, 155 aa, synthetic construct
  source 1..155: note = Deduced amino acid sequence TaCIPK14-4D of KO-3
MANRGKILMERYELGRLLGKGTFGKVHYARSLESNRSVAIKMLDKEKVLKVGLSEQIRREVTTMRLVAHKNIVQLHEVMATRNKIYFVMEYVKAVSSLTRLQRVASSQRVLHISISSSSSVQWITATAKACITGISSWRTCSWMRMRTLRSRILD

SEQ ID NO: 129 - protein, 95 aa, synthetic construct; identical to SEQ ID NO: 120
  source 1..95: note = Deduced amino acid sequence TaCIPK14-4A of KO-4

SEQ ID NO: 130 - protein, 65 aa, synthetic construct
  source 1..65: note = Deduced amino acid sequence TaCIPK14-4B of KO-4
MANRGKILMERYELGRLLGKGTFGKVHYARSLESNQSVAIKMLDKEKVLKVGLSEQIRREVTTRR

SEQ ID NO: 131 - protein, 72 aa, synthetic construct
  source 1..72: note = Deduced amino acid sequence TaCIPK14-4D of KO-4
MANRGKILMERYELGRLLGKGTFGKVHYARSLESNRSVAIKMLDKEKVLKVGLSEQIRREVVGGTQEHCSAS

SEQ ID NO: 132 - protein, 95 aa, synthetic construct; identical to SEQ ID NO: 120
  source 1..95: note = Deduced amino acid sequence TaCIPK14-4A of KO-5

SEQ ID NO: 133 - protein, 95 aa, synthetic construct; identical to SEQ ID NO: 124
  source 1..95: note = Deduced amino acid sequence TaCIPK14-4B of KO-5

SEQ ID NO: 134 - protein, 72 aa, synthetic construct; identical to SEQ ID NO: 131
  source 1..72: note = Deduced amino acid sequence TaCIPK14-4D of KO-5

(19) United States
(12) Patent Application Publication
(10) Pub. No.: US 2025/0101450 A1
(43) Pub. Date: Mar. 27, 2025

(54) Method for Improving Wheat Resistance To Fusarium Head Blight (FHB) By Genome Editing

(71) Applicant: Northwest A&F University, Xianyang, Shaanxi (CN)

(72) Inventors: Jun Guo, Xianyang, Shaanxi (CN); Fuxin He, Xianyang, Shaanxi (CN); Zhensheng Kang, Xianyang, Shaanxi (CN); Huilin Sun, Xianyang, Shaanxi (CN); Xingxuan Bai, Xianyang, Shaanxi (CN); Ce Wang, Xianyang, Shaanxi (CN); Yanfeng Wang, Xianyang, Shaanxi (CN)

(73) Assignee: Northwest A&F University, Xianyang, Shaanxi (CN)

(21) Appl. No.: 18/558,619

(22) PCT Filed: Dec. 29, 2022
(86) PCT No.: PCT/CN2022/143089

§ 371 Date: Nov. 2, 2023

(30) Foreign Application Priority Data
Jun. 15, 2022 (CN) 202210674184.4

(51) Int. Cl. C12N 15/82; C12N 9/22; C12N 15/11

(52) U.S. Cl. CPC C12N 15/8282; C12N 9/22; C12N 15/11; C12N 15/8205; C12N 15/8213; C12N 2310/20

(57) ABSTRACT

A method for improving wheat resistance to Fusarium head blight (FHB) by genome editing is provided, and further provided is use of TaCIPK14 protein or related biomaterial thereto in the regulation of plant resistance to FHB; the TaCIPK14 protein is TaCIPK14-4A protein (SEQ ID NO: 4), TaCIPK14-4B protein (SEQ ID NO: 5), and/or TaCIPK14-4D protein (SEQ ID NO: 6). TaCIPK14 knockout mutant plants are successfully obtained by simultaneously editing three alleles of TaCIPK14 in wheat using CRISPR-Cas9 gene editing technology. The TaCIPK14 knockout mutant shows resistance to inoculation with FHB pathogens, and the results of evaluation of major agronomic traits confirm that the TaCIPK14 knockout mutant reserves the major agronomic traits.

### CROSS-REFERENCE TO RELATED APPLICATION

This patent application is a national phase application of PCT application No. PCT/CN2022/143089, filed on Dec. 29, 2022, which claims the benefit and priority of Chinese Patent Application No. 202210674184.4 filed with the China National Intellectual Property Administration on Jun. 15, 2022, both of which are incorporated by reference herein in their entirety as part of the present application.

### REFERENCE TO SEQUENCE LISTING

A computer readable XML file entitled “GWPCTP20230705602_seqlist”, that was created on Sep. 18, 2023, with a file size of about 174,597 bytes, contains the sequence listing for this application, has been filed with this application, and is hereby incorporated by reference in its entirety.

### TECHNICAL FIELD

The present disclosure relates to the technical field of breeding of disease-resistant varieties, and in particular to a method for improving wheat resistance to Fusarium head blight (FHB) by genome editing.

### BACKGROUND

Fusarium head blight (FHB) is a fungal disease that severely disrupts global wheat production, causing billions of dollars in losses during wheat production each year (McMullen et al., 2012). Since 2000, wheat FHB epidemics have become more severe due to global warming, and the number of years of wheat FHB epidemics has significantly increased, with yield losses as high as 70% in some fields (Yerkovich et al., 2017). Since 2010, the area of wheat FHB has been increasing, and according to data from the National Center for Agricultural Technology Extension (NCATE), the annual average area of wheat FHB in China from 2011-2015 amounted to 82 million mu. Particularly, in 2012, the incidence of wheat FHB was extensive and the extent was historically rare, with an incidence area of 140 million mu (https://www.natesc.org.cn/). Chemical control is currently major means for controlling wheat FHB in the field. However, the long-term use of a single agent leads to resistance of pathogen to fungicides, and breeding disease-resistant varieties is the most economical, effective, and environmentally friendly strategy to prevent FHB. Although breeders have improved wheat resistance to FHB through traditional breeding approaches, there is still a lack of stable FHB resistant varieties (Wang et al., 2020). Therefore, it is still the main task to mine for genes associated with FHB resistance and to breed stable resistant varieties in the breeding of wheat with FHB resistance. The enhancement of wheat disease resistance should be balanced with wheat yield in order to meet the needs of wheat production and population growth.

Susceptibility genes(S) are necessary for pathogens and hosts to establish affinity interactions, and at the same time, they are the key for pathogens to successfully invade the host. It has become an important tool to utilize plant susceptibility genes to improve plant disease resistance in the breeding process, and the gene editing technology Clustered Regularly Interspaced Short Palindromic Repeats (CRISPR) can realize non-transgenic editing of susceptibility genes, thus reducing the potential risks associated with the transfer of exogenous genes. The editing of susceptibility genes can be utilized to rapidly improve the disease resistance of crops, thus facilitating the development of disease-resistance breeding.

In summary, the current technical problems are as following.

Wheat susceptibility gene resources are scarce and cannot meet the current breeding needs, and the traditional genetic improvement method of screening susceptibility genes is time-consuming, so how should we quickly screen to obtain wheat susceptibility genes and improve the wheat varieties?

As editing of susceptibility genes in the wheat variety Fielder was found to significantly improve disease resistance in wheat, can susceptibility genes be edited in major domestic cultivars, and can edition of susceptibility genes in cultivars improve their disease resistance?

Editing of wheat susceptibility genes can significantly increase wheat resistance to pathogens. However, editing of susceptibility genes may lead to the occurrence of wheat immune overload and depletion of nutrients during its own growth and development, so how should one balance wheat resistance to disease and maintenance of normal growth and development?

### SUMMARY

It is an object of the present disclosure to provide a method for improving wheat resistance to FHB in by genome editing.

In a first aspect, the present disclosure is drawn to use of TaCIPK14 protein or a related biomaterial thereto in regulation of plant resistance to FHB.

The TaCIPK14 protein is a TaCIPK14-4A protein, a TaCIPK14-4B protein and/or a TaCIPK14-4D protein;

the TaCIPK14-4A protein is selected from the group consisting of
(A1) a protein having the amino acid sequence of SEQ ID NO: 4;
(A2) a wheat-derived protein having the amino acid sequence of SEQ ID NO: 4 with one or more substitutions and/or deletions and/or additions of amino acid residues, and having the same function;
(A3) a wheat-derived protein having at least 99%, at least 95%, at least 90%, at least 85%, or at least 80% identity to the amino acid sequence of any of (A1)-(A2) and having the same function; and
(A4) a fusion protein obtained by attaching a protein tag to an N- and/or C-terminus of the protein of any one of (A1)-(A3);
the TaCIPK14-4B protein is selected from the group consisting of
(B1) a protein having the amino acid sequence of SEQ ID NO: 5;
(B2) a wheat-derived protein having the amino acid sequence of SEQ ID NO: 5 with one or more substitutions and/or deletions and/or additions of amino acid residues, and having the same function;
(B3) a wheat-derived protein having at least 99%, at least 95%, at least 90%, at least 85%, or at least 80% identity to the amino acid sequence of any of (B1)-(B2) and having the same function; and
(B4) a fusion protein obtained by attaching a protein tag to an N- and/or C-terminus of the protein of any one of (B1)-(B3);
the TaCIPK14-4C protein is selected from the group consisting of
(C1) a protein having the amino acid sequence of SEQ ID NO: 6;
(C2) a wheat-derived protein having the amino acid sequence of SEQ ID NO: 6 with one or more substitutions and/or deletions and/or additions of amino acid residues, and having the same function;
(C3) a wheat-derived protein having at least 99%, at least 95%, at least 90%, at least 85%, or at least 80% identity to the amino acid sequence of any of (C1)-(C2) and having the same function; and
(C4) a fusion protein obtained by attaching a protein tag to an N- and/or C-terminus of the protein of any one of (C1)-(C3).

In the above description of proteins, the term ‘protein tag’ refers to a polypeptide or protein expressed in fusion with a target protein using the technology of DNA recombination in vitro to facilitate expression, detection, tracing and/or purification of the target protein. The protein tag is selected from the group consisting of a Flag-tag, a His-tag, an MBP-tag, an HA-tag, a myc-tag, a GST-tag and/or a SUMO-tag.

In the above description of proteins, the term ‘identity’ refers to the identity of the amino acid sequence. Identity of amino acid sequences can be determined using alignment search tools on the internet, such as BLAST of the NCBI homepage website. For example, this can be done in Advanced BLAST 2.1 by using blastp as program, with an Expect value being 10, all Filters being OFF, BLOSUM62 as Matrix, and Gap existence cost, Per residue gap cost and Lambda ratio set to 11, 1 and 0.85 (default values), respectively, and performing search to calculate the identity of a pair of amino acid sequences, so that the identity (%) is obtained.

In the above description of proteins, ‘at least 95% identity’ may be at least 96%, 97%, 98% identity. ‘At least 90% identity’ may be at least 91%, 92%, 93%, 94% identity. ‘At least 85% identity’ may be at least 86%, 87%, 88%, 89% identity. ‘At least 80% identity’ may be at least 81%, 82%, 83%, or 84% identity.

The related biomaterial includes a nucleic acid molecule capable of expressing the TaCIPK14 protein, or an expression cassette, a recombinant vector, a recombinant microorganism or a transgenic cell line containing the nucleic acid molecule.

The expression cassette is a DNA capable of expressing TaCIPK14 in a host cell, and the DNA may include not only a promoter that initiates transcription of TaCIPK14, but also a terminator that terminates transcription of the TaCIPK14 gene. Furthermore, the expression cassette may also include an enhancer sequence. Promoters that can be used in the present disclosure include, but are not limited to constitutive promoters, tissue-, organ- and development-specific promoters, and inducible promoters. Exemplary promoters include, but are not limited to the Ubiqutin promoter (pUbi); the constitutive 35S promoter of cauliflower mosaic virus (CaMV); the trauma-inducible promoter from tomato, leucine aminopeptidase (“LAP”, Chao et al. (1999) Plant Physiol 120:979-992); the chemically inducible promoter, pathogenesis-related 1 (PR1) from tobacco, which is induced by salicylic acid and BTH (benzothiadiazole-7-thioglycolic acid S-methyl ester); tomato protease inhibitor II promoter (PIN2) or LAP promoter (both inducible with cockroach jasmonate); heat shock promoter (U.S. Pat. No. 5,187,267); tetracycline-inducible promoter (U.S. Pat. No. 5,057,422); embryo-specific promoter, such as seed-specific promoter pF128 from foxtail millet (CN101063139B (CN Patent NO: 200710099169.7)), embryonic storage protein-specific promoters (e.g., promoters for legumin from common bean, napin, oleosin, and beta conglycinin from soybean (Beachy et al. (1985) EMBO J. 4:3047-3053)). They can be used alone or in combination with other plant promoters. All references cited herein are incorporated in their entirety. Suitable transcriptional terminators include, but are not limited to the Nopaline Synthase terminator (NOS terminator), the CaMV 35S terminator, the tml terminator, the pea rbcS E9 terminator, and mannopine synthase (Mas) or octopine synthase (Ocs) terminators (see, e.g., Odell et al. (I⁹⁸⁵) Nature 313:810; Rosenberg et al. (1987) Gene, 56:125; Guerineau et al. (1991) Mol. Gen. Genet, 262:141; Proudfoot (1991) Cell, 64:671; Sanfacon et al. Genes Dev. Cell, 2:1261; Munroe et al. (1990) Gene, 91:151; Ballad et al. (1989) Nucleic Acids Res. 17:7891; Joshi et al. (1987) Nucleic Acid Res., 15:9627).

A recombinant expression vector containing the TaCIPK14 gene expression cassette is constructed. The plant expression vectors utilized may be binary Agrobacterium vectors or Gateway system vectors, etc., such as pBin438, pCAMBIA1302, pCAMBIA2301, pCAMBIA1301, pCAMBIA1300, pBI121, pGWB411, pGWB412, pGWB405, pCAMBIA1391-Xa or pCAMBIA1391-Xb. When TaCIPK14 is used to construct recombinant expression vectors, any one of the enhancement, constitutive, tissue-specific, or inducible promoters, such as the CaMV) 35S promoter, the Ubiqutin promoter (pUbi), etc., can be added before its transcription start nucleotide etc., which can be used alone or in combination with other plant promoters; furthermore, when the gene of the present disclosure is used to construct plant expression vectors, enhancers including translational enhancers and transcriptional enhancers can also be used. These enhancers can be located in the region where the ATG start codons is located or a neighboring region, provided that the region shares the same reading frame with the coding sequence in order to ensure the correct translation of the whole sequence. The sources of the translation control signals and start codons are wide-ranging and can be natural or synthetic. Translation initiation regions can be derived from transcription initiation regions or structural genes.

In order to facilitate the identification and screening of transgenic plant cells or plants, the plant expression vectors used can be processed, such as incorporating genes encoding enzymes or luminescent compounds that can produce color changes (GUS genes, luciferase genes, etc.), antibiotic-resistant markers (gentamicin markers, kanamycin markers, etc.), or chemical reagent-resistant markers (e.g., herbicide-resistant genes), etc., which can be expressed in plants.

In the use, the vector may be a plasmid, a cosmid, a phage or a viral vector.

In the use, the microorganisms may be yeast, bacteria, algae or fungi. The bacteria may be selected from the group consisting of genera Escherichia, Erwinia, Agrobacterium (e.g. Agrobacterium rhizogenes EHA105), Flavobacterium, Alcaligenes, Pseudomonas, and Bacillus.

In the use, the regulation of plant resistance to FHB may be manifested as a reduction in expression and/or activity of the TaCIPK14 protein and an increase in plant resistance to FHB in the plant.

Herein, the reduction in expression and/or activity of the TaCIPK14 protein is preferably a simultaneous reduction in expression and/or activity of the TaCIPK14-4A protein, the TaCIPK14-4B protein and the TaCIPK14-4D protein, if the TaCIPK14 gene is knocked out or knocked down simultaneously on the wheat A genome, B genome and D genome.

In a second aspect, the present disclosure is drawn to use of a substance capable of causing a reduction in expression and/or activity of the TaCIPK14 protein in a plant in any one of the following:
(D1) Improvement of plant resistance to FHB; and
(D2) Breeding plant varieties with improved resistance to FHB.

The TaCIPK14 protein is selected from the group consisting of the TaCIPK14-4A protein, the TaCIPK14-4B protein, and/or the TaCIPK14-4D protein, as previously described.

In some embodiments, the substance may be an RNA interference fragment, an RNA interference vector, a homologous recombinant fragment, a homologous recombinant vector, or a gene editing tool capable of causing a decrease in the expression and/or activity of the TaCIPK14 protein in the plant.

In a specific embodiment of the present disclosure, the substance is a CRISPR/CRISPR-associated protein 9 (Cas9) system, where the target sequence of the gRNA includes SEQ ID NO: 7 (gRNA1) and/or SEQ ID NO: 8 (gRNA2). Expression cassettes for gRNA1 and gRNA2 were constructed in tandem by digestion and ligation into a gene editing vector VK006 to produce a specific recombinant plasmid VK005-06-g1g2. The specific recombinant plasmid VK005-06-g1g2 contains a gene coding for a Cas9 protein and genes coding for the gRNA1 and the gRNA2.

In a third aspect, the present disclosure is drawn to a method for increasing plant resistance to FHB.

The method for increasing plant resistance to FHB in the present disclosure includes causing a decrease in expression and/or activity of the TaCIPK14 protein in a recipient plant.

The TaCIPK14 protein includes the TaCIPK14-4A protein, the TaCIPK14-4B protein, and the TaCIPK14-4D protein, as previously described.

In a fourth aspect, the present disclosure is drawn to a method for breeding plant varieties with improved resistance to FHB.

The method for breeding plant varieties with improved resistance to FHB in the present disclosure includes causing a reduction in expression and/or activity of the TaCIPK14 protein in the recipient plant.

The TaCIPK14 protein includes the TaCIPK14-4A protein, the TaCIPK14-4B protein, and the TaCIPK14-4D protein, as previously described.

The methods in the third and fourth aspects may be achieved by hybridization or by genetic modification.

In the third and fourth aspects, causing a reduction in expression and/or activity of the TaCIPK14 protein in a recipient plant can be achieved by methods known in the art, such as RNA interference, homologous recombination, genome targeted editing, and the like.

In some embodiments, this can be achieved specifically by inhibiting expression of TaCIPK14.

In some embodiments, this can be achieved specifically by knocking out TaCIPK14, the knocking out including knocking out entire or a partial fragment of TaCIPK14.

In some embodiments, this can be achieved specifically by silencing TaCIPK14.

In some embodiments, this can be achieved specifically by gene editing of TaCIPK14.

In specific embodiments of the present disclosure, the gene editing is realized with the aid of a CRISPR/Cas9 system. In the CRISPR/Cas9 system, the guide RNA (gRNA) comprises gRNA1 and gRNA2, and the target sequences of gRNA1 and gRNA2 are shown in SEQ ID NO: 7 and SEQ ID NO: 8, respectively. The gene editing is achieved by introducing into the recipient plant (e.g., wheat) a specific DNA molecule containing the gene encoding the Cas9 protein and the gene encoding the gRNA. The gene editing is achieved by introducing a recombinant plasmid containing the specific DNA molecule in the recipient plant (e.g., wheat).

In a fifth aspect, the present disclosure claims a method for breeding a transgenic plant with improved resistance to FHB.

The method for breeding a transgenic plant with improved resistance to FHB in the present disclosure may include the steps of: inhibiting expression of a nucleic acid molecule capable of expressing the TaCIPK14 protein in a recipient plant, and obtaining a transgenic plant with improved resistance to FHB.

The TaCIPK14 protein includes the TaCIPK14-4A protein as previously described, the TaCIPK14-4B protein as previously described, and/or the TaCIPK14-4D protein as previously described.

In the method, it is understood that the transgenic plant include not only a first and a second generation of the transgenic plant, but also their offspring. In the case of the transgenic plant, the gene can be propagated in the species, or it can be transferred into other varieties of the same species, including in particular commercial varieties, using conventional breeding techniques. The transgenic plant includes seeds, healing tissues, complete plants and cells.

In some embodiments, inhibition of expression of a nucleic acid molecule capable of expressing the TaCIPK14 protein in the recipient plant may be achieved by introducing an RNA interference fragment, a RNA interference vector, a homologous recombinant fragment, a homologous recombinant vector, or a gene editing tool, etc., into the recipient plant.

In a specific embodiment of the present disclosure, the gene editing tool is a CRISPR/Cas9 system, where the target sequences of the gRNA include SEQ ID NO: 7 (gRNA1) and/or SEQ ID NO: 8 (gRNA2). The expression cassettes for gRNA1 and gRNA2 were constructed in tandem by enzymatic ligation into gene editing vector VK006 to generate a recombinant plasmid VK005-06-g1g2. The specific recombinant plasmid VK005-06-g1g2 contains a gene encoding Cas9 protein and genes encoding the gRNA1 and the gRNA2.

In the method, after attainment of the transgenic plant by inhibiting expression of a nucleic acid molecule capable of expressing the TaCIPK14 protein in the recipient plant, the method may further include a step of screening for a gene-edited plant from the transgenic plant, the gene-edited plant having higher resistance to FHB than the recipient plant.

In the method, after attainment of the transgenic plant by inhibiting expression of a nucleic acid molecule capable of expressing the TaCIPK14 protein in the recipient plant, the method may further include steps of self-crossing the transgenic plant, obtaining self-crossed progeny, and screening a gene-edited plant from the self-crossed progeny, the gene-edited plant having higher resistance to Fhb than the recipient plant.

Screening of the gene-edited plant may specifically include screening of a transgenic plant in which gene editing occurs and TaCIPK14 expression is simultaneously suppressed on the A, B and D genomes. If the TaCIPK14 gene is not edited on the A, B and D genomes at the same time, the method further include steps of: crossing the plant (e.g., wheat) in which editing of the TaCIPK14 gene occurs on the A, B or D genomes, self-crossing the progeny of a crossed plant, and screening for a transgenic plant (e.g., wheat) in which TaCIPK14 is edited in A, B and D genomes simultaneously and the expression of TaCIPK14 is suppressed.

Self-crossing the transgenic plant (e.g., wheat) may specifically include self-crossing a transgenic plant (e.g., wheat) in which gene editing occurs and TaCIPK14 expression is suppressed. Screening the gene-edited plant (e.g. wheat) from the self-cross progeny may specifically include screening the transgenic plant (e.g. wheat) from the self-cross progeny in which gene editing occurs and TaCIPK14 expression is suppressed.

The gene-edited plant (e.g., wheat) is a plant (e.g., wheat) that satisfies the following conditions: the A genome, the B genome, and the D genome are all mutated in the target region and are all homozygous.

The gene-edited plant (e.g., wheat) is a plant (e.g., wheat) that satisfies the following conditions: the A genome, the B genome, and the D genome are all mutated in the target region, are all homozygous, and does not carry a vector sequence.

The progeny obtained by self-crossing homozygous mutant plants in which all three copies of the TaCIPK14 gene in the A, B, and D genomes of a plant (e.g., wheat) are knocked out at the same time are known as gene-edited plants.

In all of the above aspects, the nucleic acid molecule capable of expressing the TaCIPK14 protein may be nucleic acid molecule A, nucleic acid molecule B, and/or nucleic acid molecule C.

The nucleic acid molecule A is capable of expressing the TaCIPK14-4A protein; the nucleic acid molecule B is capable of expressing the TaCIPK14-4B protein; and the nucleic acid molecule C is capable of expressing the TaCIPK14-4D protein.

The nucleic acid molecule A may be any of the following:
(a1) A DNA molecule set forth in SEQ ID NO: 1;
(a2) A DNA molecule hybridized with the DNA molecule defined in (a1) under stringent conditions and encoding the TaCIPK14-4A protein;
(a3) A DNA molecule having at least 99%, at least 95%, at least 90%, at least 85%, or at least 80% identity to any of the DNA sequences defined in (a1)-(a2) and encoding the TaCIPK14-4A protein;

The nucleic acid molecule B may be any of the following:
(b1) A DNA molecule set forth in SEQ ID NO: 2;
(b2) A DNA molecule hybridized with the DNA molecule defined in (b1) under stringent conditions and encoding the TaCIPK14-4B protein;
(b3) A DNA molecule having at least 99%, at least 95%, at least 90%, at least 85%, or at least 80% homology to any of the DNA sequences defined in (b1)-(b2) and encoding the TaCIPK14-4B protein;

The nucleic acid molecule C may be any of the following:
(c1) A DNA molecule set forth in SEQ ID NO: 3;
(c2) A DNA molecule hybridized with the DNA molecule defined in (c1) under stringent conditions and encoding the TaCIPK14-4D protein;
(c3) A DNA molecule having at least 99%, at least 95%, at least 90%, at least 85%, or at least 80% homology to any of the DNA sequences defined in (c1)-(c2) and encoding the TaCIPK14-4D protein.

For the above nucleic acid molecules, the stringent conditions may be as follows: 50° C., hybridized in a mixture of 7% sodium dodecyl sulfate (SDS), 0.5M Na₃PO₄ and 1 mM EDTA, rinsed at 50° C., 2×SSC, 0.1% SDS; and may also be: 50° C., hybridized in a mixture of 7% SDS, 0.5M Na₃PO₄ and 1 mM EDTA, rinsed at 50° C. 1×SSC, 0.1% SDS; or: 50° C., hybridized in a mixture of 7% SDS, 0.5M Na₃PO₄ and 1 mM EDTA, rinsed at 50° C., 0.5×SSC, 0.1% SDS; or: 50° C., hybridized in a mixture of 7% SDS, 0.5M Na₃PO₄ and 1 mM EDTA, rinsed at 50° C., 0.1×SSC, 0.1% SDS; or: 50° C., hybridized in a mixture of 7% SDS, 0.1×SSC, 0.1% SDS; also available as: hybridized at 50° C. in a mixture of 7% SDS, 0.5M Na₃PO₄ and 1 mM EDTA, rinsed at 65° C. in 0.1×SSC, 0.1% SDS; also available as: hybridized at 65° C. in a solution of 6×SSC, 0.5% SDS, followed by 2×SSC, 0.1% SDS and 1×SSC, 0.1% SDS each to wash the membrane once.

In the above description of nucleic acid molecules, the term ‘identity’ refers to the identity of the amino acid sequence. Identity of amino acid sequences can be determined using alignment search tools on the internet, such as BLAST of the NCBI homepage website. For example, this can be done in Advanced BLAST 2.1 by using blastp as program, with an Expect value being 10, all Filters being OFF, BLOSUM62 as Matrix, and Gap existence cost, Per residue gap cost and Lambda ratio set to 11, 1 and 0.85 (default values), respectively, and performing search to calculate the identity of a pair of amino acid sequences, so that the identity (%) is obtained.

In the above description of nucleic acid molecules, ‘at least 95% identity’ may be at least 96%, 97%, 98% identity. ‘At least 90% identity’ may be at least 91%, 92%, 93%, 94% identity. ‘At least 85% identity’ may be at least 86%, 87%, 88%, 89% identity. ‘At least 80% identity’ may be at least 81%, 82%, 83%, or 84% identity.

In all of the above aspects, the plant may be a monocotyledonous plant or a dicotyledonous plant.

In some embodiments, the monocotyledon may be a graminaceous plant.

In some other embodiments, the graminaceous plant may be a plant of genus Triticum.

More specifically, the plant of genus Triticum may be Triticum aestivum.

The varieties of Triticum aestivum may specifically be Fielder, Jimai 22, Zhengmai 7698, Xinong 979, Bainong 207, Xinmai 26 and the like. The wheat described in the present disclosure is not limited to the above mentioned varieties.

In all of the foregoing aspects, the pathogen of the FHB may be Fusarium graminearum.

In a specific embodiment of the present disclosure, the pathogen of the FHB is the wild-type strain of F. graminearum PH-1.

In a sixth aspect, the present disclosure claims a transgenic plant with improved resistance to FHB obtained by the method in the fifth aspect hereinbefore.

In particular, the transgenic plant may be a genetically modified plant, seed or tissue, etc.

### BRIEF DESCRIPTION OF THE DRAWINGS

FIGS. 1A-1C shows a schematic diagram of the cDNA sequence alignment analysis of three copies of TaCIPK14 (SEQ ID NO: 1-3) provided in an example of the present disclosure.

FIG. 2 shows a schematic diagram of a protein sequence alignment analysis of three copies of TaCIPK14 (SEQ ID NOs: 4-6) provided in an example of the present disclosure, where * indicates the kinase catalyzation domain and arrows indicate the activation loop.

FIG. 3 shows a schematic diagram of editing vector VK005-06 for editing the wheat gene provided in an example of the present disclosure.

FIG. 4 shows a schematic diagram of the editing vector VK005-06-g1g2 for editing the wheat gene TaCIPK14 provided in an example of the present disclosure.

FIGS. 5A-5F show schematic diagrams of the results of sequencing analysis of TaCIPK14 To transgenic plants provided in an example of the present disclosure, where FIG. 5A-5C are schematic diagrams of the results of TaCIPK14 To transgenic plants T₀-4 and T₀-12 (SEQ ID NOS: 37-45) where editing occurs at the target sequence gRNA2; and FIGS. 5D-5F are schematic diagrams of the results of TaCIPK14 To transgenic plant T₀-21 (SEQ ID NOs: 46-51) where editing occurs at the target sequence gRNA1.

FIG. 6 shows a nucleotide alignment between the homzygous mutant with three copies of TaCIPK14 knocked out simultaneously in the wheat A, B, and D genomes and wild-type (SEQ ID NOs: 52-101) in an example of the present disclosure, where//indicates omissions.

FIG. 7 shows the deduced amino acid sequence alignment between the homozygous mutant with the three copies of TaCIPK14 knocked out simultaneously in the wheat A, B, and D genomes and the wild-type (SEQ ID NOs: 102-134) in an example of the present disclosure, where // indicates omissions, and * indicates termination.

FIGS. 8A-8B show a schematic diagram of the TaCIPK14 gene-edited plant provided in an example of the present disclosure inoculated with FHB pathogens exhibiting resistance, where FIG. 8A shows the incidence of wild-type plants and TaCIPK14 gene-edited plants after infection with FHB pathogens; and FIG. 8B shows the statistical analysis of the number of diseased spikelets in wild-type plants and TaCIPK14 gene-edited plants after FHB pathogen infestation.

### DETAILED DESCRIPTION OF THE EMBODIMENTS

The following examples facilitate a better understanding of the disclosure, but do not limit the disclosure. The experimental methods in the following examples are conventional methods, if not otherwise specified. The test materials used in the following examples are, unless otherwise specified, purchased from a conventional biochemical reagent store. The quantitative tests in the following examples are set up for three repetitions, and the results are averaged.

The plant CRISPR/Cas9 gene editing vector is VK005-06, which is a product from Beijing Viewsolid Biotech Co., Ltd. and contains the wheat U6 promoter TaU6 for initiating small guide RNA (sgRNA), Cas9p mimics the graminaceous plant genes with higher GC content at the 5′ end, and is a designed and synthesized gene with optimized codons for plants. The primers used in this disclosure were synthesized by Xi′an Tsingke Zexi Biotechnology Co. Ltd.

For the quantitative tests in the following examples, three replicate experiments are set up and the results are averaged.

Wheat variety Fielder (referred to as “Fielder” in the literature): Bai X, Zhan G, Tian S, et al. Transcription factor BZR2 activates chitinase Cht20. 2 transcription to confer resistance to wheat stripe rust [J]. Plant Physiology, 2021, 187 (4): 2749-2762. Fielder is assigned as WT, and the corresponding sequence in the A genome is assigned as WT-A, the corresponding sequence in the B genome is assigned as WT-B, and the corresponding sequence in the D genome is assigned as WT-D.

### Example 1: Preparation of Recombinant Plasmids

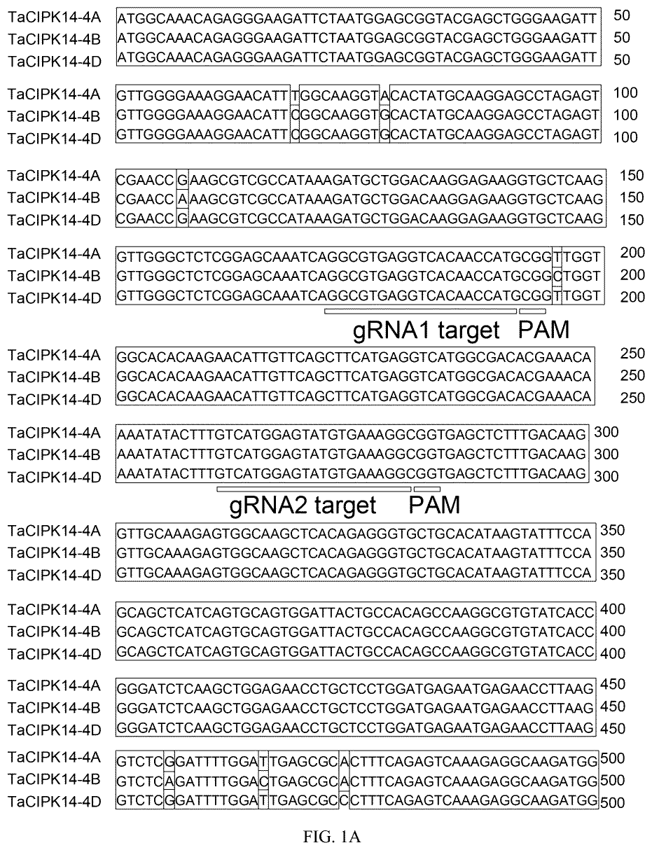

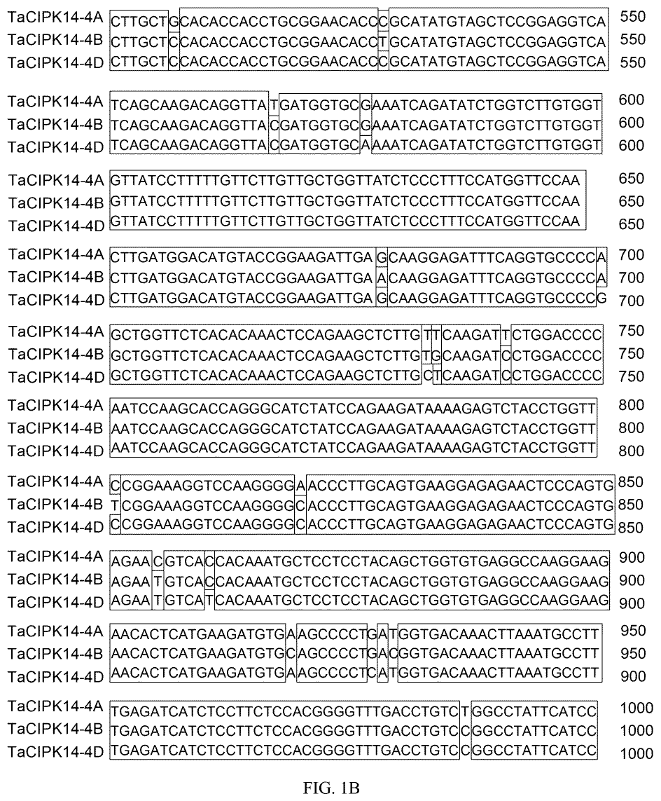

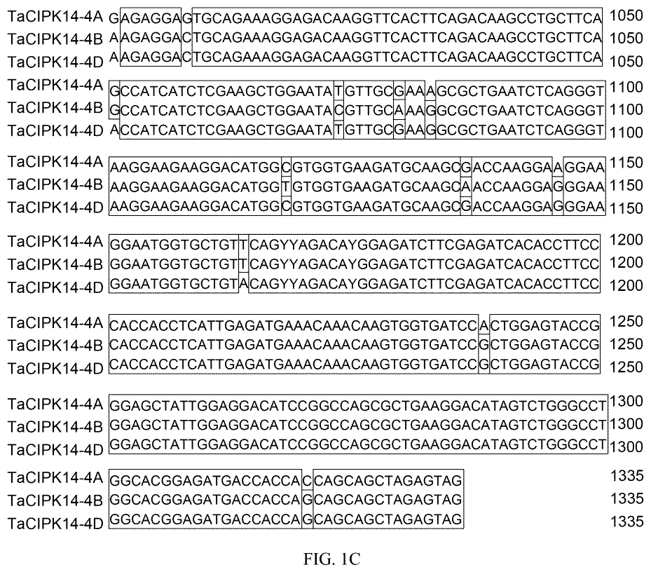

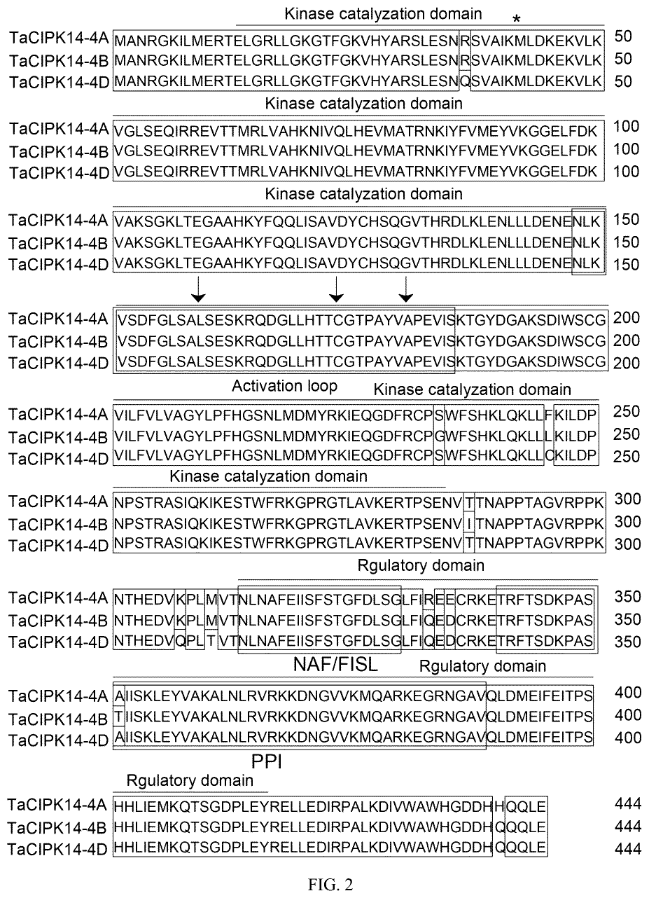

The genomic sequences and coding sequences of the TaCIPK14 gene on chromosomes A, B, and D were amplified from the DNA and cDNA of the wheat variety Fielder. The nucleotide sequence of TaCIPK14 in the A genome was denoted by TaCIPK14-4A, and the nucleotide sequence of TaCIPK14 in the B genome was denoted by TaCIPK14-4B. The nucleotide sequence of TaCIPK14 in D genome was denoted by TaCIPK14-4D. The amplification results showed that the TaCIPK14 gene was 1335 bases in length and did not contain introns. In the wheat Fielder genome, the nucleotide sequence of the TaCIPK14 gene in the A genome (corresponding to the A chromosome group) is set forth in SEQ ID NO: 1 of the sequence listing (encoding the protein of SEQ ID NO: 4), which is 100% identical to that in the Ensembl Plants database (http://plants.ensembl.org/index.html); the nucleotide sequence of the TaCIPK14 gene in the B genome (corresponding to the B chromosome group) is set forth in SEQ ID NO: 2 of the sequence listing (encoding the protein of SEQ ID NO: 5), which is 100% identical to that of wheat in the Ensembl Plants database; the nucleotide sequence of the TaCIPK14 gene in the B genome (corresponding to the B chromosome group) is set forth in SEQ ID NO: 3 of the sequence listing (encoding the protein of SEQ ID NO: 6), which is 100% identical to that of wheat in the Ensembl Plants database. The schematic diagram of the sequence alignment analysis of the cDNA sequences of the three copies of TaCIPK14 is shown in FIGS. 1A-1C. The schematic diagram of the protein sequence alignment analysis of the three copies of TaCIPK14 is shown in FIG. 2.

Through the website E-CRISPR (http://www.e-crisp.org/), common guide RNA (gRNA) sequences the TaCIPK14 gene on A, B and D genomes were found, and the 20 bp sequences in front of the Protospacer Adjacent Motif (PAM) were set as gRNA sequences. In the present Example, two of the sgRNAs were taken for the knockdown experiments as an example, the sequences were gRNA1: 5′-GGCGTGAGGTCACAACCATG-3′ (SEQ ID NO: 7) and gRNA2: 5′-GTCATGGAGTATGTGAAA GG-3′ (SEQ ID NO: 8).

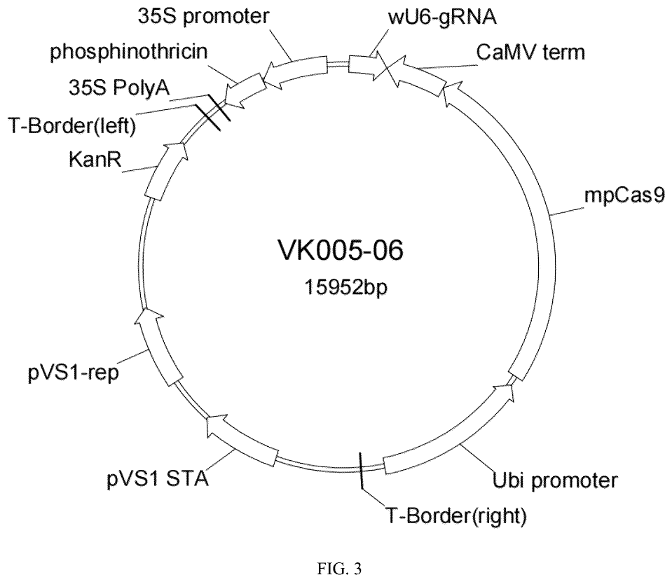

VK005-6-gRNA1 recombinant plasmid and VK005-6-gRNA2 recombinant plasmid were constructed according to the instructions of the Plant Cas9/gRNA Plasmid Construction Kit (Beijing Viewsolid Biotech Co. Ltd.). The schematic diagram of gene editing vector VK005-06 is shown in FIG. 3. The structure of VK005-6-gRNA1 recombinant plasmid is described as a recombinant plasmid obtained by replacing positions 392-411 of the VK005-06 vector with the DNA fragment of SEQ ID NO: 7. The structure of VK005-6-gRNA2 recombinant plasmid is described as the recombinant plasmid obtained by replacing positions 392-411 of the VK005-06 vector with the DNA fragment of SEQ ID NO: 8.

The gRNA1 and gRNA2 were tandemly ligated together to construct a recombinant plasmid VK005-06-g1g2, and the specific steps included the followings. The recombinant plasmid VK005-6-gRNA1 was double-digested with AscI and Spel, and a short band (wU6: 510 bp) was recovered by running gels, which was ligated into the vector of recombinant plasmid VK005-6-gRNA2 double-digested with AscI and AvrII. The ligated product was transformed into Escherich coli, and then cultured on LB (kan) plates until the clone grew. Then a single clone was picked for sequencing with the sequencing primer:
VK005-6-R: 5′-GATGAAGTGGACGGAAGGAAGGAG-3′ (SEQ ID NO: 9).

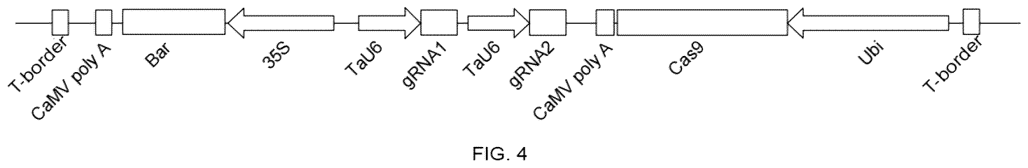

The sequencing results showed that the target sequences of sgRNA, gRNA1 and gRNA2 were detected, and TaU6 promoter sequences were all detected upstream of the target sequences. The sequencing results indicated that the sgRNA expression cassette was successfully constructed and assembled into the VK005-06 binary expression vector, which proved the successful construction of the CRISPR/Cas9 gene editing vector for TaCIPK14. The schematic diagram of the editing vector VK005-06-g1g2 for editing wheat TaCIPK14 gene is shown in FIG. 4.

The structure of the VK005-06-g1g2 vector is described as a recombinant plasmid obtained by replacing positions 392-411 of the VK005-06 vector with the DNA fragment of SEQ ID NO: 7, and replacing positions 864-883 of the VK005-06 vector with the DNA fragment of SEQ ID NO: 8 meanwhile.

### Example 2: Obtaining Gene-Edited Wheat by Agrobacterium-Mediated Genetic Transformation Using gRNA1 and gRNA2

### I. Agrobacterium-Mediated Genetic Transformation of Wheat

The gene editing recombinant plasmid VK005-06-g1g2 constructed in Example 1 was used to transform immature young embryos of the wheat variety Fielder by Agrobacterium-mediated genetic transformation, followed by differentiation, screening, regeneration, and rooting, and ultimately regenerated plants were obtained. Wheat genetic transformation was accomplished by the Genetic Transformation Platform of the State Key Laboratory of Crop Stress Biology for Arid Areas, Northwest Agriculture and Forestry University (NWAFSU), which can be commercially available to the public for the transformation operation.

### II. Detection of Gene Editing

### 1. Identification of Regenerated Plants of to Generation

The test plants were: 22 To generation regenerated plants obtained in step I, and Fielder (as a reference plant for the regenerated plants). Genomic DNA was extracted from the leaves of the test plants and amplified by PCR using three specific primer pairs. The three specific primer pairs were: primer pairs consisting of TaCIPK14-A-F and TaCIPK14-A-R (the target sequences of the primer pairs in the wheat genomic DNA are set forth in SEQ ID NO: 1, where the gRNA1 and gRNA2 of the TaCIPK14 gene are located in the wheat A genome), TaCIPK14-B-F and TaCIPK14-B-R consisting of primer pairs (the target sequences of the primer pairs in wheat genomic DNA are set forth in SEQ ID NO: 2, in which gRNA1 and gRNA2 of the TaCIPK14 gene are located in the wheat B genome), TaCIPK14-D-F and TaCIPK14-D-R (the target sequences of the primer pairs in wheat genomic DNA are set forth in SEQ ID NO: 3, where gRNA1 and gRNA2 of the TaCIPK14 gene are located in the wheat D genome). The sequences of the three specific primer pairs are:

TaCIPK14-A-F:
(SEQ ID NO: 10)
5′-GATTGTTGGGGAAAGGAACATTT-3′;
TaCIPK14-A-R:
(SEQ ID NO: 11)
5′-TTGTCTCCTTTCTGCACTCCTCTC-3′;
TaCIPK14-B-F:
(SEQ ID NO: 12)
5′-CGCAGTCCATTTGAATTATAGCCA-3′;
TaCIPK14-B-R:
(SEQ ID NO: 13)
5′-ATTCAGCGCCTTTGCAACG-3′;
TaCIPK14-D-F:
(SEQ ID NO: 14)
5′-AAGACATGAGAGTGTTGTTCACTGTG-3′;
TaCIPK14-D-R:
(SEQ ID NO: 15)
5′-AGCTGTAGGAGGAGCATTTGTGA-3′.

2. Sequencing of the Recovered PCR Amplification Products after Step 1.

The amplification products were purified and subjected to first-generation sequencing (Sanger sequencing), and the sequencing results were compared with the A, B, and D genomic sequences of the TaCIPK14 gene in Fielder. If sequence alignment shows double peaks at the target site, it is indicated heterozygous or bi-allelic mutations occurs at the target site, if there are base deletions or insertions at the target site, it is indicated homozygous mutations occurs at the target site, and if the nucleotide sequences of the target site is consistent with that of the PCR amplification products of Fielder, the regenerated plants are wild-type. The PCR products with mutations were ligated into T-Simple vector and transformed into E. coli, and the positive clones obtained were sequenced by colony PCR amplification using universal primers to clarify the mutation mode of TaCIPK14.

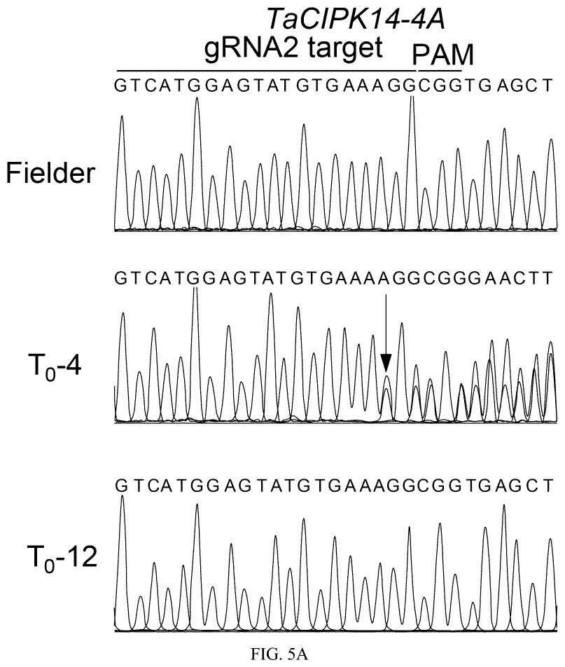

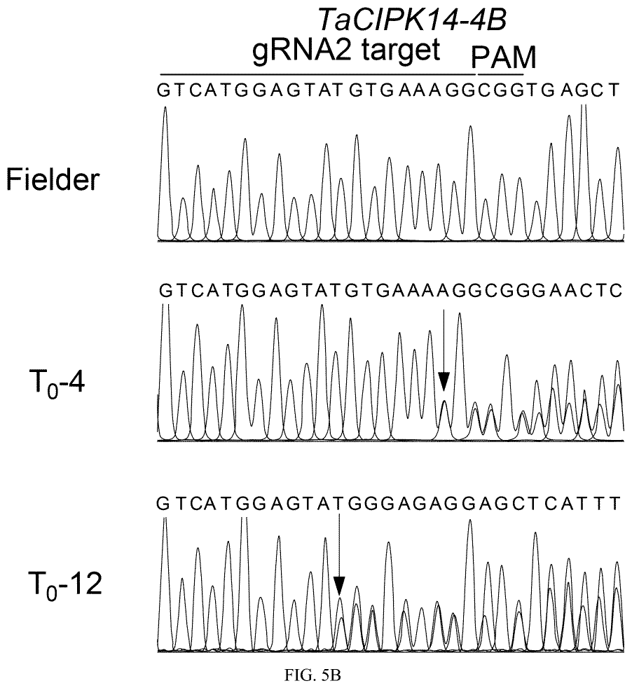

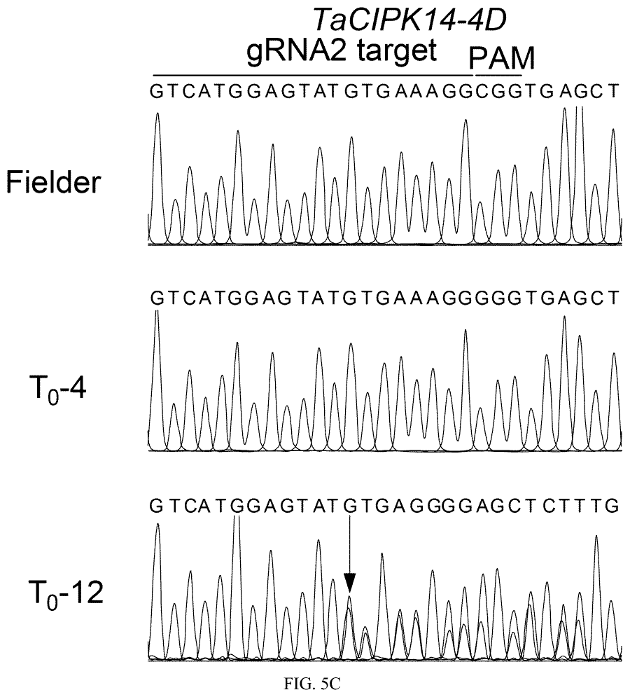

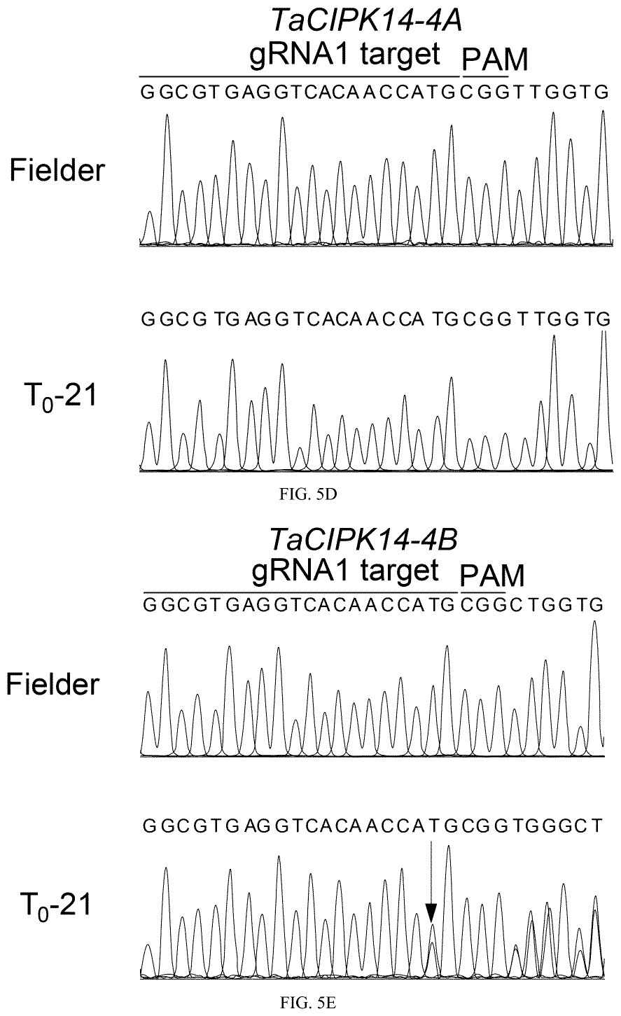

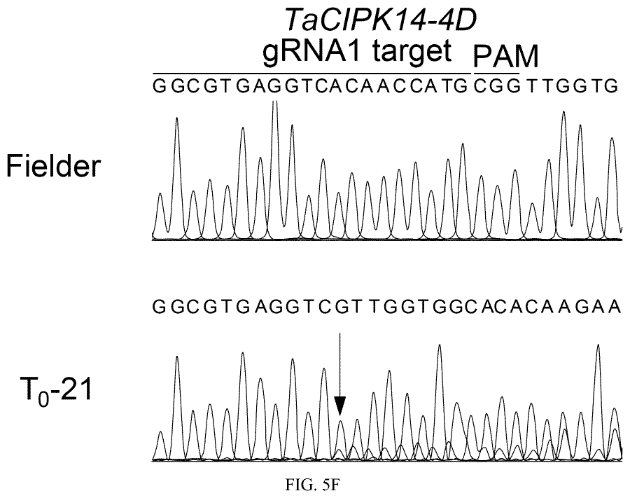

If the PCR amplification product of the regenerated plant is only one and the nucleotide sequence is consistent with that of the PCR amplification product of Fielder, the regenerated plant is wild-type. If the PCR amplification product of the regenerated plant is of two kinds, one of which is consistent with the nucleotide sequence of the PCR amplification product of Fielder, and the other of which has a mutation (the mutation includes a deletion, insertion, or substitution of one or more nucleotides) compared to the nucleotide sequence of the PCR amplification product of Fielder, the regenerated plant is heterozygous. If the PCR amplification product of the regenerated plant is of two kinds, both of which have mutations (the mutations include deletions, insertions or substitutions of one or more nucleotides) compared to the nucleotide sequence of the PCR amplification product of Fielder, the regenerated plant is bi-allelic mutant. If the PCR amplification product of the regenerated plant is of one kind and has a mutation (the mutation includes a deletion, insertion or substitution of one or more nucleotides) compared to the nucleotide sequence of the PCR amplification product of Fielder, the regenerated plant is a homozygous mutant. If the PCR amplification products of the regenerated plant are of three or more kinds, the regenerated plant is chimeric. Heterozygous, bi-allelic mutant, homozygous mutant, and chimeric plants were collectively referred to as edited plants. Of the 22 regenerated plants, 3 were edited plants (13.6%), and 19 were wild-type (86.4%). The results of the sequencing analysis of TaCIPK14 T₀ transgenic plants are shown schematically in FIGS. 5A-5F.

The genotypes of the three gene-edited strains based on the target sequence A, B, and D genomes, and the types of mutations based on the target sequences are shown in Table 1.

TABLE 1
Genotypes of T₀ gene edited strains

| T₀ generation | Genotypes based on | | Mutation types based on |
|---|---|---|---|
| plant number | target sequences | | target sequences |
| T₀-4 | A | +1 | heterozygous mutant |
| | B | +1 | heterozygous mutant |
| | D | wt | wild type |
| T₀-12 | A | wt | wild type |
| | B | −5 | heterozygous mutant |
| | D | −1, −5 | chimeric mutant |
| T₀-21 | A | wt | wild type |
| | B | −89 | heterozygous mutant |
| | D | −11 | heterozygous mutant |

Note:
− stands for deletion, −5 stands for deletion of 5 nucleotides, and so on; + stands for insertion, +1 stands for insertion of 1 nucleotide, and so on.

The TaCIPK14 gene in one homologous chromosome in the A genome of T₀-4 was wild-type (SEQ ID NO: 1), and the TaCIPK14 gene in one homologous chromosome was the mutant gene obtained by inserting one base between nucleotides 276-277 (i.e., SEQ ID NO: 1 at positions 276-277) in the wild-type gene; The TaCIPK14 gene in one homologous chromosome in the B genome was wild-type (SEQ ID NO: 2), and the TaCIPK14 gene in one homologous chromosome was a mutant gene obtained by inserting one base between nucleotides 276-277 (i.e., positions 276-277 of SEQ ID NO: 2) in the wild-type gene; The TaCIPK14 gene in two homologous chromosomes in the D genome was wild-type (SEQ ID NO: 3). With the exception of wild type, all of the above mutant types of the gene resulted in a code-shifting mutation leading to a loss of function of the TaCIPK14 protein.

The TaCIPK14 gene in two homologous chromosomes in the A genome of T₀-12 was wild-type (SEQ ID NO: 1); the TaCIPK14 gene in one homologous chromosome in the B genome was wild-type (SEQ ID NO: 2) and in one homologous chromosome was a mutant gene obtained by deleting nucleotides at positions 272-276 (i.e., positions 272-276 of SEQ ID NO: 2) from the wild-type gene; the TaCIPK14 gene in the D genome was a chimeric mutation, in which one genotype was wild-type (SEQ ID NO: 3), one genotype was a mutation obtained by deleting the nucleotide at position 278 (i.e., position 278 of SEQ ID NO: 3) from the wild-type gene, and one genotype was a mutant gene obtained by deleting nucleotides at positions 274-278 (i.e., positions 274-278 of SEQ ID NO: 3) from the wild-type gene. With the exception of wild type, all of the above genotypes resulted in a code-shifting mutation leading to a loss of function of the TaCIPK14 protein.

The TaCIPK14 gene in two homologous chromosomes in the A genome of T₀-21 was wild-type (SEQ ID NO: 1); the TaCIPK14 gene in one homologous chromosome in the B genome was wild-type (SEQ ID NO: 2), and the TaCIPK14 gene in one homologous chromosome was a mutant gene obtained by deleting nucleotides at positions 190-278 (i.e., positions 190-278 of SEQ ID NO: 2) from the wild-type gene; the TaCIPK14 gene in one homologous chromosome in the D genome was wild-type (SEQ ID NO: 3), and in one homologous chromosome was a mutant gene obtained by deleting nucleotides at positions 184-194 (i.e., positions 184-194 of SEQ ID NO: 3) from the wild-type gene; With the exception of wild type, all of the above mutant types of the gene resulted in a code-shifting mutation leading to a loss of function of the TaCIPK14 protein.

### 2. Characterization of T₁ Generation Plants

To generation plants were taken for self-crossing to obtain T₁ generation seeds, and the T₁ generation seeds were cultivated to obtain T₁ generation plants.

Individual T₁ generation plants were characterized according to the method of step 1.

The results of each identification are shown in Table 2.

TABLE 2
Genetic profile of T₁ generation plants

| | number | | Number of | Number of | Number of |
|---|---|---|---|---|---|
| | of | | wild-type | heterozygous | homozygous |
| | plants | | plants | mutant plants | mutant plants |
| T₁ generation plants | 21 | A | 4 | 10 | 7 |
| from T₀ generation | | B | 6 | 10 | 5 |
| plant T₀-4 | | D | 21 | 0 | 0 |
| T₁ generation plants | 14 | A | 14 | 0 | 0 |
| from T₀ generation | | B | 4 | 5 | 5 |
| plant T₀-12 | | D | 0 | 8 | 6 |
| T₁ generation plants | 36 | A | 36 | 0 | 0 |
| from T₀ generation | | B | 11 | 19 | 6 |
| plant T₀-21 | | D | 6 | 22 | 8 |

It was shown that the D genome of T₀-4, the A genome of T₀-12, and the A genome of T₀-21 of the unedited TaCIPK14 were stably inherited to T₁, and did not produce mutations; for the edited heterozygous mutant of TaCIPK14, segregation in T₁ obtained by strict self-crossing was in accordance with Mendelian inheritance; for the edited chimeric mutant of TaCIPK14, segregation in T₁ obtained by strict self-crossing did not conform to Mendelian inheritance.

T₁ generation plants that are of the mutation type ‘A genome homozygous mutant, B genome homozygous mutant and D genome wild type’ based on target sequence were selected from the T₁ generation plants of the T₀ generation plant T₀-4, T₁ generation plants that are of the mutation type ‘A genome wild type, B genome homozygous mutant and D genome homozygous mutant’ based on target sequence were selected from the T₁ generation plants of the T₀ generation plant T₀-12, and T₁ generation plants that are of the mutation type ‘A genome wild type, B genome homozygous mutant and D genome homozygous mutant’ based on target sequence were selected from the T₁ generation plants of the T₀ generation plant T₀-21. The selected T₁ generation plants were self-crossed and the resultant seeds were harvested. T₂ generation seeds were cultivated to obtain T₂ generation plants.

3. Creation of a Homozygous Mutant Strain with Simultaneous Knockout of Three Copies of TaCIPK14 in the A, B and D Genomes of Wheat

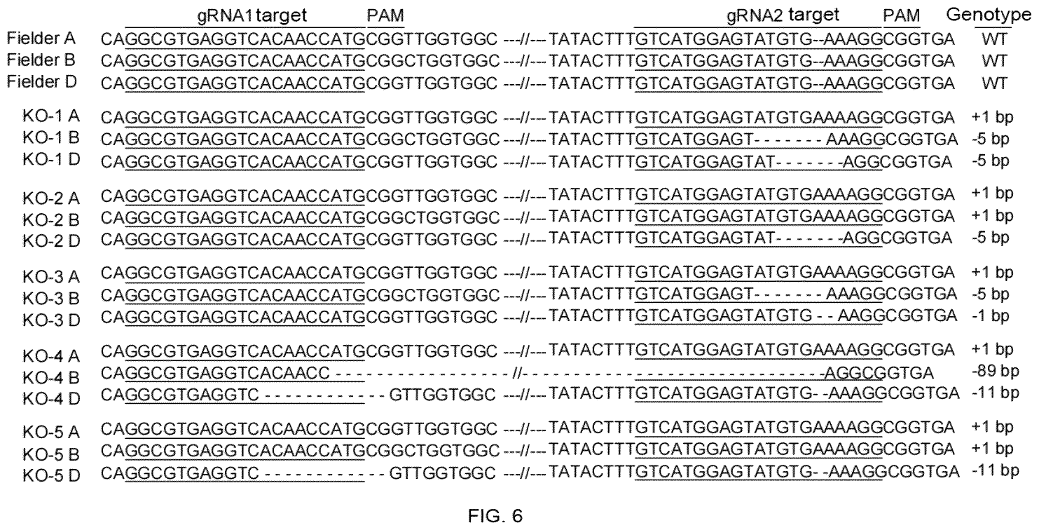

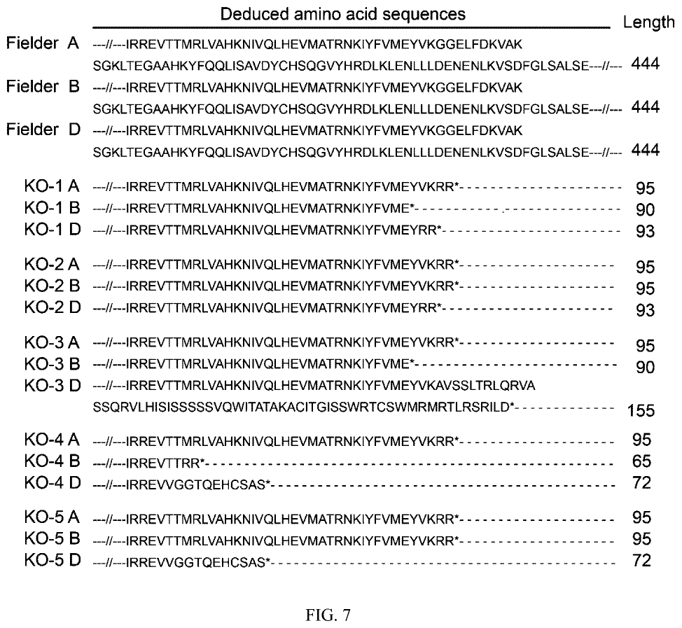

In order to obtain homozygous mutant strains with three copies of TaCIPK14 knocked out simultaneously in the A, B and D genomes of wheat, T₂ generation plants that are of the mutation type ‘A genome homozygous mutant, B genome homozygous mutant and D genome wild type’ based on the target sequence were selected as the male parent, and T₂ generation plants that are of the mutation type ‘A genome wild type, B genome homozygous mutant and D genome homozygous mutant’ based on the target sequence were selected as the female parent. The selected T₂ generation plants were crossed to obtain F₁ plants. Each F₁ plant was characterized according to the method in step 1. F₁ plants with a target sequence-based mutation type ‘A genome heterozygous mutant, B genome heterozygous mutant and D genome heterozygous mutant’ were selected for self-crossing to obtain F₂ plants. Each of the F₂ plants was characterized according to the method in step 1. The F₂ plants with the mutation type ‘A genome-homozygous mutant, B genome-homozygous mutant and D genome-homozygous mutant’ based on the target sequences were selected, and the selected F₂ plants were self-crossed to obtain F₃ plants, which were the homozygous mutant strain with all three copies of TaCIPK14 knocked out simultaneously in the wheat A, B, and D genomes. In the process of obtaining homozygous mutant strain with three copies of TaCIPK14 knocked out simultaneously in wheat A, B and D genomes, the genotypic identification results of each generation of plants are shown in Table 3. The nucleotide alignment between homozygous mutant and wild-type genes with three copies of TaCIPK14 knocked out simultaneously in wheat A, B and D genomes is shown in FIG. 6. The deduced amino acid sequences of the three copies of TaCIPK14 in the wheat A, B and D genomes were aligned with those of the wild-type gene as shown in FIG. 7, indicating that the knock out of the three copies of TaCIPK14 in the wheat A, B and D genomes in the homozygous mutant phenotype resulted in code-shifting mutation, which led to a loss of function of the TaCIPK14 protein. It was shown that the homozygous mutant strain with TaCIPK14 mutation in T₂ generation could be inherited to F₁ and F₂ generation plants by crossing, and for the strict self-crossing of TaCIPK14 heterozygous mutant strain in F₁ generation plants to obtain the homozygous mutant strain with simultaneous knockout of all three copies of TaCIPK14 in wheat A, B, and D genomes, the segregation conformed to Mendelian inheritance. Homozygous mutant strains with simultaneous knockout of three copies of TaCIPK14 in wheat A, B and D genomes, including KO-1, KO-2, KO-3, KO-4 and KO-5, were available at F₂ generation.

TABLE 3
Genetic profile of T₂, F₁, F₂ generation plants

| | | | | | | | | | | | Genotype analysis of F2 plant | | | | | |
|---|---|---|---|---|---|---|---|---|---|---|---|---|---|---|---|---|
| | | | | | | | | | | | | Homo- | | | | Number |
| | | | | | | | | Genotype | | | | zygous | | | | homo- |
| | | | | | | | | analysis of F1 | | | | three- | | | | zygous |
| Hybrid parent | | | | | | | | plant | | | | copy | | | | three- |
| | | | Mutation | | | | Mutation | F₁ | Mutation | | | homo- | | | Mutation | copy |
| Male | Genotypes | | types | Female | Genotypes | | types | Num- | types | | Num- | zygous | Genotypes | | types | homo- |
| parent | based on | | based on | parent | based on | | based on | ber | based on | | ber | mutant | based on | | based on | zygous |
| plant | target | | target | plant | target | | target | of | target | | of F₂ | plant | target | | target | mutant |
| number | sequences | | sequence | number | sequences | | sequence | plants | sequence | | plants | Number | sequences | | sequence | plants |
| T₂-4-1 | A | +1 | Homo- | T₂-12-12 | A | w t | wild | 10 | A | Hetero- | 81 | KO-1 | A | 1 | homozygous | 2 |
| | | | zygous | | | | type | | | zygous | | | | | mutant | |
| | | | mutant | | | | | | | mutant | | | B | −5 | Homozygous | |
| | | | | | | | | | | | | | | | mutant | |
| | B | +1 | Homo- | | B | −5 | Homo- | | B | Hetero- | | | D | −5 | Homozygous | |
| | | | zygous | | | | zygous | | | zygous | | | | | mutant | |
| | | | mutant | | | | mutant | | | mutant | | | | | | |
| | | | | | | | | | | | | KO-2 | A | 1 | Homozygous | 2 |
| | | | | | | | | | | | | | | | mutant | |
| | D | w t | wild | | D | −5 | Homo- | | D | Hetero- | | | B | 1 | Homozygous | |
| | | | type | | | | zygous | | | zygous | | | | | mutant | |
| | | | | | | | mutant | | | mutant | | | D | −5 | Homozygous | |
| | | | | | | | | | | | | | | | mutant | |
| T₂-4-13 | A | +1 | Homo- | T₂-12-1 | A | w t | wild | 10 | A | Hetero- | 87 | KO-3 | A | 1 | Homozygous | 1 |
| | | | zygous | | | | type | | | zygous | | | | | mutant | |
| | | | mutant | | | | | | | mutant | | | | | | |
| | B | +1 | Homo- | | B | −5 | Homo- | | B | Hetero- | | | B | −5 | Homozygous | |
| | | | zygous | | | | zygous | | | zygous | | | | | mutant | |
| | | | mutant | | | | mutant | | | mutant | | | | | | |
| | D | wt | wild | | D | −1 | Homo- | | D | Hetero- | | | D | −1 | Homozygous | |
| | | | type | | | | zygous | | | zygous | | | | | mutant | |
| | | | | | | | mutant | | | mutant | | | | | | |
| T₂-4-1 | A | +1 | Homo- | T₂-21-12 | A | w t | wild | 8 | A | Hetero- | 44 | KO-4 | A | 1 | Homozygous | 1 |
| | | | zygous | | | | type | | | zygous | | | | | mutant | |
| | | | mutant | | | | | | | mutant | | | | | | |
| | B | +1 | Homo- | | B | −89 | Homo- | | B | Hetero- | | | B | −89 | Homozygous | |
| | | | zygous | | | | zygous | | | zygous | | | | | mutant | |
| | | | mutant | | | | mutant | | | mutant | | | | | | |
| | D | w t | wild | | D | −11 | Homo- | | D | Hetero- | | | D | −11 | Homozygous | |
| | | | type | | | | zygous | | | zygous | | | | | mutant | |
| | | | | | | | mutant | | | mutant | | | | | | |
| T₂-4-13 | A | +1 | Homo- | T₂-21-2 | A | w t | wild | 9 | A | Hetero- | 69 | KO-5 | A | 1 | Homozygous | 2 |
| | | | zygous | | | | type | | | zygous | | | | | mutant | |
| | | | mutant | | | | | | | mutant | | | | | | |
| | B | +1 | Homo- | | B | −89 | Homo- | | B | Hetero- | | | B | 1 | Homozygous | |
| | | | zygous | | | | zygous | | | zygous | | | | | mutant | |
| | | | mutant | | | | mutant | | | mutant | | | | | | |
| | D | w t | wild | | D | −11 | Homo- | | D | Hetero- | | | D | −11 | Homozygous | |
| | | | type | | | | zygous | | | zygous | | | | | mutant | |
| | | | | | | | mutant | | | mutant | | | | | | |

### III. Off-Target Analysis of CRISPR/Cas9

The possible off-target sites of the sgRNA1 target of TaCIPK14 gene were predicted according to the prediction software CasOT (http://eendb.zfgenetics.org/casot/), and primers were designed based on the flanking sequences of the possible off-target sites: a primer pair consisting of g1off1-F and g1off1-R, a primer pair consisting of g1off2-F and g1off2-R, a primer pair consisting of g1off3-F and g1off3-R, and a primer pair consisting of g1off4-F and g1off4-R. The possible off-target sites of the sgRNA2 target of TaCIPK14 were also predicted according to the prediction software CasOT, and primers were designed according to the flanking sequences of the possible off-target sites: a primer pair consisting of g2off5-F and g2off5-R, a primer pair consisting of g2off6-F and g2off6-R, and a primer pair consisting of g2off7-F and g2off7-R.

g1off1-F:
(SEQ ID NO: 16)
5′-CGGAGTTCGGGACCTCGGTG-3′;
g1off1-R:
(SEQ ID NO: 17)
5′-TCGCCTTCCTCCTCCTCCTGCC-3′.
g1off2-F:
(SEQ ID NO: 18)
5′-ACCTCCTCCTCCTTCCTGTCGTCTT-3′;
g1off2-R:
(SEQ ID NO: 19)
5′-TACGGGGTTGCTGGCACTTG-3′.
g1off3-F:
(SEQ ID NO: 20)
5′-CACGGAGACGCCCAGAGAC-3′;
g1off3-R:
(SEQ ID NO: 21)
5′-CGCACGGTAGGAGCAACGG-3′.
g1off4-F:
(SEQ ID NO: 22)
5′-TATTTCCTCCATCTTGCTCTCTTCC-3′;
g1off4-R:
(SEQ ID NO: 23)
5′-CAAGTGTCTACTCTCTCCATCCACAA-3′.
g2off5-F:
(SEQ ID NO: 24)
5′-CGTGTGCCTGCTTTTTGCTG-3′;
g2off5-R:
(SEQ ID NO: 25)
5′-CGCCAGTGTTGATGTGAGATTGT-3′.
g2off6-F:
(SEQ ID NO: 26)
5′-GGGGAGGATGTGGGCAGAT-3′;
g2off6-R:
(SEQ ID NO: 27)
5′-CGCAACGAGGACCAACACTTTA-3′.
g2off7-F:
(SEQ ID NO: 28)
5′-CTCATCCATTTTTATCACAACCTTAGA-3′;
g2off7-R:
(SEQ ID NO: 29)
5′-GCTGACCGCCAGACCCACT-3′.

(1) T₂ generation of TaCIPK14 gene-edited plants (T₂-4-1, T₂-21-2, and T₂-12-12) and homozygous mutant F₃ plants (KO-1, KO-2, KO-3, KO-4, and KO-5) with three copies of TaCIPK14 knocked out at the same time in the wheat A, B, and D genomes were taken and their leaves were extracted for genomic DNA.
(2) With the genomic DNA extracted in step (1) as a template, the primer pair consisting of g1off1-F and g1off1-R, the primer pair consisting of g1off2-F and g1off2-R, the primer pair consisting of g1off3-F and g1off3-R, the primer pair consisting of g1off4-F and g1off4-R, the primer pair consisting of g2off5-F and g2off5-R, the primer pair consisting of g2off6-F and g2off6-R, and the primer pair consisting of g2off7-F and g2off7-R were used for PCR amplification.
(3) The PCR amplification product obtained in step (2) was sequenced.

Off-target sites are shown in Table 4.

TABLE 4
List of off-target sites

| | | | | Number of |
|---|---|---|---|---|
| | | | | differential |
| | | | | nucleotides |
| | Off- | | | compared |
| gRNA | target | Location of off- | Nucleotide sequence of | to the target |
| targets | site | target sites | off-target sites | site |
| gRNA1 | OFF1 | chr2D: 139522328: | aGCtcGAG_GTCACAACCAT | 3 |
| | | 139524351 | G-GGG (SEQ ID NO: 30) | |
| | OFF2 | chr3D: 156768495: | aGCtaGAG_GTCACAACCAT | 3 |
| | | 156770518 | G-GGG (SEQ ID NO: 31) | |
| | OFF3 | chr5D: 534426169: | aGCtcGAG_GTCACAACCAT | 3 |
| | | 534428192 | G-GGG (SEQ ID NO: 32) | |
| | OFF4 | chr7A: 176673733: | cGaGTGAG_GTCgCAACCAT | 3 |
| | | 176675756 | G-CGG (SEQ ID NO: 33) | |
| gRNA2 | OFF5 | chr2B: 130489523: | gGTTCTgT_CCTTCtCTGCAA- | 3 |
| | | 130491546 | TGG (SEQ ID NO: 34) | |
| | OFF6 | chr2B: 676945246: | aaTTtTCT_CCTTCgCTGCAA- | 3 |
| | | 676947269 | AGG (SEQ ID NO: 35) | |
| | OFF7 | chr | tGcTCTCT_aCTTCACTGCAA- | 3 |
| | | 6a: 42782254: | TGG (SEQ ID NO: 36) | |
| | | 42784277 | | |

It was shown that for the seven predicted potential off-target sites mentioned above, no off-targets were detected in T₂ generation TaCIPK14 gene-edited plants (T₂-4-1, T₂-21-2, and T₂-12-12) and homozygous mutant F₃ plants (KO-1, KO-2, KO-3, KO-4, and KO-5) with simultaneous knockout of all three copies of TaCIPK14 in the wheat A, B, and D genomes. i.e., No off-target effects of gRNA1 and gRNA2 were detected at potential off-target sites.

### Example 3: Analysis of Wheat FHB Resistance

The test plants were Fielder plants, and homozygous mutant strain with three copies of TaCIPK14 knocked out simultaneously in the wheat A, B, and D genomes, including KO-1, KO-4, and KO-5 prepared in Example 2.

The test plants were characterized for resistance to FHB in the following steps:
(1) F. graminearum wild-type strain PH-1, the causal agent of wheat FHB, was used for the culture of conidial spore, which was donated by Mr. Liu Huiquan from the College of Plant Protection, Northwest Agriculture and Forestry University, and was recorded in the paper “Li Yimin et al., Functional characterization of the HDACs genes in Fusarium graminearum. Northwest Agriculture and Forestry University, 2010, Master's Degree thesis”, which is available to the public from the applicant, and may be used only for the purpose of repeating the experiments of the present disclosure, not for any other purpose. The strain was cultured and activated on PDA solid medium, and a piece of solid with FHB strain was taken from the above medium with a disc sampler, put into 200 mL of sterilized carboxymethyl cellulose (CMC) liquid medium, then cultured at 25° C. and 180 rpm for 4-5 days with oscillation, sampled for microscopic examination, and then filtered through gauze. The spore suspension was prepared, and the concentration was 1×10⁵ spores/ml.
(2) Selection and preparation of plastic bags: transparent plastic bags with a size of 30 cm×40 cm×50 μm in length×width×thickness and plastic ties for bundling were purchased.
(3) FHB pathogen inoculation and bag moisturizing: during the wheat flowering, spore suspension prepared in step (1) were taken, 10 μL of which were dropped to the lower part of the spikelet base of the florets. At least 30 ears were inoculated for each strain, and the inoculation site of the glumes was marked with a marker. Inner side of the transparent plastic bags prepared in step (2) and pathogen-inoculated spikes were sprayed. The inoculated spikes were bagged with plastic bags, which were moderately tied at internode under the spike with plastic ties. The date of inoculation was marked on waterproof labels at inoculation row plot that was easy to observe, the moisturizing was conducted for 3 days, and then the moisturizing bag was removed.
(4) Observation, documentation and statistics: the number of diseased spikelets was surveyed and counted 21 days after inoculation, the resistance of test plants to FHB was evaluated as the average number of diseased spikelets, and the significance of differences was analyzed.

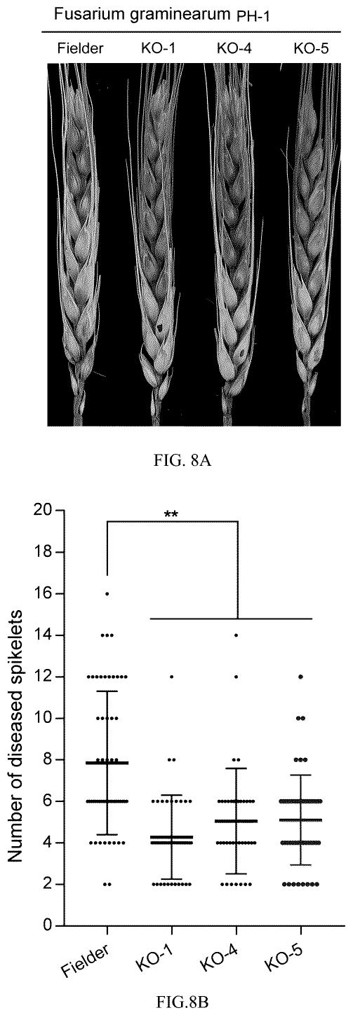

The number of diseased spikelets and the significance of differences of the test plants showed that the FHB infection of the wild-type material Fielder had expanded to the whole spike or half a spike, while the FHB infection of the homozygous mutant strain with three copies of TaCIPK14 knocked out simultaneously in the wheat A, B, and D genomes, including KO-1, KO-4, and KO-5, had expanded by only less than half a spike or very little. Statistical analysis of the significance of differences showed that the average number of diseased spikelets in inoculated spikelets was significantly lower (P<0.05) in the homozygous mutant strains (KO-1, KO-4, and KO-5) in which all three copies of TaCIPK14 were knocked out simultaneously in the wheat A, B, and D genomes, as compared to the wild-type material, Fielder. The schematic diagram of TaCIPK14 gene-edited plants inoculated with FHB pathogen showing resistance to the disease is shown in FIGS. 8A-8B. It is proved that TaCIPK14 gene-edited plants showed strong resistance to FHB pathogen.

### Example 4: Agronomic Trait Analysis

The wild-type recipient variety Fielder and the homozygous mutant strains (KO-1, KO-4 and KO-5) with three copies of TaCIPK14 knocked out simultaneously in the wheat A, B and D genomes were planted together in the artificial climate chamber at the Southeast Kiln of the College of Plant Protection, Northwest Agriculture and Forestry University. The phenotypes of the above tested edited plants and wild-type plants at seedling stage and the filling stage, main spike at the maturity stage, and grain length and width of grains at the maturity stage were observed, and the main agronomic traits were counted. The statistical results are shown in Table 5. The observation results showed that, compared with the wild-type Fielder, the homozygous mutant strains (KO-1, KO-4, and KO-5) with the three copies of TaCIPK14 simultaneously knocked out in the wheat A, B, and D genomes did not show significant changes in plant morphology at the seedling and filling stages, as well as in the morphology of the spike and the grain at the maturity stage. Statistical analyses showed that there were no significant differences in plant height, flag leaf length, spike length, grain number per spike, grain length, grain width, and thousand-grain weight in the homozygous mutant strains (KO-1, KO-4, and KO-5) in which all three copies of TaCIPK14 were knocked out simultaneously in the A, B, and D genomes of wheat, as compared to those of the wild-type Fielder. This indicates that the wheat TaCIPK14 gene is involved as a susceptibility gene in wheat susceptibility to FHB, the TaCIPK14 knockout mutant has significantly improved resistance to wheat FHB, the TaCIPK14 knockout mutant maintains major agronomic traits, and the wheat TaCIPK14 gene editing material can be used as a new germplasm material that can be used for genetic improvement of wheat resistance to FHB. The technical solution of the present disclosure provides a practical method for realizing rapid wheat breeding and improving wheat disease resistance by genetic engineering technology, which has important breeding application value and broad market application prospect.

TABLE 5
Statistical results of agronomic traits

| | | | | Grain | | | Thousand- |
|---|---|---|---|---|---|---|---|
| | Plant | Flag leaf | Spike | number | Grain | Grain | grain |
| | height | length | length | per | length | width | weight |
| | (cm) | (cm) | (cm) | spike | (cm) | (cm) | (g) |
| Fielder | 65.0 ± 6.9 | 19.8 ± 2.2 | 8.9 ± 0.8 | 29.3 ± 4.0 | 0.73 ± 0.03 | 0.33 ± 0.02 | 29.2 ± 1.4 |
| KO-1 | 63.9 ± 5.8 | 19.5 ± 1.6 | 8.8 ± 0.9 | 28.4 ± 4.6 | 0.73 ± 0.02 | 0.32 ± 0.02 | 28.7 ± 1.5 |
| KO-4 | 65.5 ± 5.1 | 20.4 ± 2.1 | 8.9 ± 1.0 | 27.3 ± 5.0 | 0.72 ± 0.03 | 0.33 ± 0.02 | 29.5 ± 1.5 |
| KO-5 | 65.2 ± 6.3 | 20.0 ± 1.9 | 8.7 ± 1.0 | 26.7 ± 3.9 | 0.72 ± 0.04 | 0.33 ± 0.02 | 29.8 ± 1.3 |

### INDUSTRIAL APPLICATION

In the present disclosure, TaCIPK14 gene-edited plants of are obtained by Agrobacterium-mediated genetic transformation using CRISPR-Cas9 gene editing technology. The TaCIPK14 gene-edited plants are characterized for disease resistance, and it is found that the TaCIPK14 gene-edited plants have significantly improved disease resistance. Wheat TaCIPK14 gene is involved as a susceptibility gene in wheat susceptibility to FHB, and the TaCIPK14-edited wheat plants have significantly improved resistance to FHB, which can be used as new germplasm material for genetic improvement of wheat resistance to FHB.

The present disclosure utilizes CRISPR-Cas9 gene editing technology to simultaneously edit three alleles of TaCIPK14 in wheat, and after the detection of possible off-target genes predicted by Cas-Offinder, it is found that there is no possibility of off-targeting to other genes in TaCIPK14 gene-edited plants. Therefore, TaCIPK14 knockout mutant plants are successfully obtained. The TaCIPK14 knockout mutant showed resistance to inoculation with FHB pathogen, and the results of the evaluation of the major agronomic traits confirm that the TaCIPK14 knockout mutant still maintained the major agronomic traits. As a result, wheat plants with the potential for wheat FHB control are obtained.

1. (canceled)

2. (canceled)

3. (canceled)

4. (canceled)

5. (canceled)

6. A method for increasing plant resistance to FHB, comprising a step of causing a decrease in expression and/or activity of TaCIPK14 protein in a recipient plant;
wherein the TaCIPK14 protein is a TaCIPK14-4A protein, a TaCIPK14-4B protein and/or a TaCIPK14-4D protein;
the TaCIPK14-4A protein is selected from the group consisting of
(A1) a protein having the amino acid sequence of SEQ ID NO: 4;
(A2) a wheat-derived protein having the amino acid sequence of SEQ ID NO: 4 with one or more substitutions and/or deletions and/or additions of amino acid residues, and having the same function;
(A3) a wheat-derived protein having at least 99%, at least 95%, at least 90%, at least 85%, or at least 80% identity to the amino acid sequence of any of (A1)-(A2) and having the same function; and
(A4) a fusion protein obtained by attaching a protein tag to an N- and/or C-terminus of the protein of any one of (A1)-(A3);
the TaCIPK14-4B protein is selected from the group consisting of
(B1) a protein having the amino acid sequence of SEQ ID NO: 5;
(B2) a wheat-derived protein having the amino acid sequence of SEQ ID NO: 5 with one or more substitutions and/or deletions and/or additions of amino acid residues, and having the same function;
(B3) a wheat-derived protein having at least 99%, at least 95%, at least 90%, at least 85%, or at least 80% identity to the amino acid sequence of any of (B1)-(B2) and having the same function; and
(B4) a fusion protein obtained by attaching a protein tag to an N- and/or C-terminus of the protein of any one of (B1)-(B3); and
the TaCIPK14-4C protein is selected from the group consisting of
(C1) a protein having the amino acid sequence of SEQ ID NO: 6;
(C2) a wheat-derived protein having the amino acid sequence of SEQ ID NO: 6 with one or more substitutions and/or deletions and/or additions of amino acid residues, and having the same function;
(C3) a wheat-derived protein having at least 99%, at least 95%, at least 90%, at least 85%, or at least 80% identity to the amino acid sequence of any of (C1)-(C2) and having the same function; and
(C4) a fusion protein obtained by attaching a protein tag to an N- and/or C-terminus of the protein of any one of (C1)-(C3).

7. A method for breeding a plant variety with improved resistance to FHB, comprising a step of causing a reduction in expression and/or activity of TaCIPK14 protein in a recipient plant;
wherein the TaCIPK14 protein is a TaCIPK14-4A protein, a TaCIPK14-4B protein and/or a TaCIPK14-4D protein;
the TaCIPK14-4A protein is selected from the group consisting of
(A1) a protein having the amino acid sequence of SEQ ID NO: 4;
(A2) a wheat-derived protein having the amino acid sequence of SEQ ID NO: 4 with one or more substitutions and/or deletions and/or additions of amino acid residues, and having the same function;
(A3) a wheat-derived protein having at least 99%, at least 95%, at least 90%, at least 85%, or at least 80% identity to the amino acid sequence of any of (A1)-(A2) and having the same function; and
(A4) a fusion protein obtained by attaching a protein tag to an N- and/or C-terminus of the protein of any one of (A1)-(A3);
the TaCIPK14-4B protein is selected from the group consisting of
(B1) a protein having the amino acid sequence of SEQ ID NO: 5;
(B2) a wheat-derived protein having the amino acid sequence of SEQ ID NO: 5 with one or more substitutions and/or deletions and/or additions of amino acid residues, and having the same function;
(B3) a wheat-derived protein having at least 99%, at least 95%, at least 90%, at least 85%, or at least 80% identity to the amino acid sequence of any of (B1)-(B2) and having the same function; and
(B4) a fusion protein obtained by attaching a protein tag to an N- and/or C-terminus of the protein of any one of (B1)-(B3);
the TaCIPK14-4C protein is selected from the group consisting of
(C1) a protein having the amino acid sequence of SEQ ID NO: 6;
(C2) a wheat-derived protein having the amino acid sequence of SEQ ID NO: 6 with one or more substitutions and/or deletions and/or additions of amino acid residues, and having the same function;
(C3) a wheat-derived protein having at least 99%, at least 95%, at least 90%, at least 85%, or at least 80% identity to the amino acid sequence of any of (C1)-(C2) and having the same function; and
(C4) a fusion protein obtained by attaching a protein tag to an N- and/or C-terminus of the protein of any one of (C1)-(C3).

8. A method for cultivating a transgenic plant with improved resistance to FHB, comprising steps of inhibiting expression of a nucleic acid molecule capable of expressing TaCIPK14 protein in a recipient plant, and obtaining the transgenic plant with improved resistance to FHB;
wherein the TaCIPK14 protein comprises TaCIPK14-4A protein, TaCIPK14-4B protein and/or TaCIPK14-4D protein;
the TaCIPK14-4A protein is selected from the group consisting of
(A1) a protein having the amino acid sequence of SEQ ID NO: 4;
(A2) a wheat-derived protein having the amino acid sequence of SEQ ID NO: 4 with one or more substitutions and/or deletions and/or additions of amino acid residues, and having the same function;
(A3) a wheat-derived protein having at least 99%, at least 95%, at least 90%, at least 85%, or at least 80% identity to the amino acid sequence of any of (A1)-(A2) and having the same function; and
(A4) a fusion protein obtained by attaching a protein tag to an N- and/or C-terminus of the protein of any one of (A1)-(A3).
the TaCIPK14-4B protein is selected from the group consisting of
(B1) a protein having the amino acid sequence of SEQ ID NO: 5;
(B2) a wheat-derived protein having the amino acid sequence of SEQ ID NO: 5 with one or more substitutions and/or deletions and/or additions of amino acid residues, and having the same function;
(B3) a wheat-derived protein having at least 99%, at least 95%, at least 90%, at least 85%, or at least 80% identity to the amino acid sequence of any of (B1)-(B2) and having the same function; and
(B4) a fusion protein obtained by attaching a protein tag to an N- and/or C-terminus of the protein of any one of (B1)-(B3).
and the TaCIPK14-4C protein is selected from the group consisting of
(C1) a protein having the amino acid sequence of SEQ ID NO: 6;
(C2) a wheat-derived protein having the amino acid sequence of SEQ ID NO: 6 with one or more substitutions and/or deletions and/or additions of amino acid residues, and having the same function;
(C3) a wheat-derived protein having at least 99%, at least 95%, at least 90%, at least 85%, or at least 80% identity to the amino acid sequence of any of (C1)-(C2) and having the same function; and
(C4) a fusion protein obtained by attaching a protein tag to an N- and/or C-terminus of the protein of any one of (C1)-(C3).

9. The method of claim 8, wherein inhibiting expression of a nucleic acid molecule capable of expressing TaCIPK14 protein in a recipient plant is achieved by introducing an RNA interference fragment, an RNA interference vector, a homologous recombinant fragment, a homologous recombinant vector, or a gene editing tool into the recipient plant.

10. The method of claim 9, wherein the gene editing tool is a CRISPR/Cas9 system, wherein a target sequence of the gRNA comprises SEQ ID NO: 7 and/or SEQ ID NO: 8.

11. The method of claim 8, wherein the nucleic acid molecule capable of expressing the TaCIPK14 protein is nucleic acid molecule A, nucleic acid molecule B and/or nucleic acid molecule C;
wherein the nucleic acid molecule A is capable of expressing the TaCIPK14-4A protein; the nucleic acid molecule B is capable of expressing the TaCIPK14-4B protein; and the nucleic acid molecule C is capable of expressing the TaCIPK14-4D protein;
the nucleic acid molecule A is selected from the group consisting of
(a1) A DNA molecule set forth in SEQ ID NO: 1;
(a2) A DNA molecule hybridized with the DNA molecule defined in (a1) under stringent conditions and encoding the TaCIPK14-4A protein; and
(a3) A DNA molecule having at least 99%, at least 95%, at least 90%, at least 85%, or at least 80% identity to any of the DNA sequences defined in (a1)-(a2) and encoding the TaCIPK14-4A protein;
the nucleic acid molecule B is selected from the group consisting of
(b1) A DNA molecule set forth in SEQ ID NO: 2;
(b2) A DNA molecule hybridized with the DNA molecule defined in (b1) under stringent conditions and encoding the TaCIPK14-4B protein; and
(b3) A DNA molecule having at least 99%, at least 95%, at least 90%, at least 85%, or at least 80% homology to any of the DNA sequences defined in (b1)-(b2) and encoding the TaCIPK14-4B protein;
and the nucleic acid molecule C is selected from the group consisting of
(c1) A DNA molecule set forth in SEQ ID NO: 3;
(c2) A DNA molecule hybridized with the DNA molecule defined in (c1) under stringent conditions and encoding the TaCIPK14-4D protein; and
(c3) A DNA molecule having at least 99%, at least 95%, at least 90%, at least 85%, or at least 80% homology to any of the DNA sequences in (c1)-(c2) and encoding the TaCIPK14-4D protein.

12. The method of claim 8, wherein the plant is a monocotyledon or a dicotyledon.

13. The method of claim 12, wherein the dicotyledon is a plant of genus Triticum.

14. The method of claim 13, wherein the plant of the genus Triticum is Triticum aestivum.

15. The method of claim 8, wherein pathogen of the FHB is Fusarium graminearum.

16. The method of claim 15, wherein the pathogen of the FHB is a wild-type strain F. graminearum PH-1.

17. (canceled)

18. The method of claim 11, wherein inhibiting expression of a nucleic acid molecule capable of expressing TaCIPK14 protein in a recipient plant is achieved by introducing an RNA interference fragment, an RNA interference vector, a homologous recombinant fragment, a homologous recombinant vector, or a gene editing tool into the recipient plant.

19. The method of claim 12, wherein inhibiting expression of a nucleic acid molecule capable of expressing TaCIPK14 protein in a recipient plant is achieved by introducing an RNA interference fragment, an RNA interference vector, a homologous recombinant fragment, a homologous recombinant vector, or a gene editing tool into the recipient plant.

20. The method of claim 12, wherein the gene editing tool is a CRISPR/Cas9 system, wherein a target sequence of the gRNA comprises SEQ ID NO: 7 and/or SEQ ID NO: 8.

21. The method of claim 12, the nucleic acid molecule capable of expressing the TaCIPK14 protein is nucleic acid molecule A, nucleic acid molecule B and/or nucleic acid molecule C;
wherein the nucleic acid molecule A is capable of expressing the TaCIPK14-4A protein; the nucleic acid molecule B is capable of expressing the TaCIPK14-4B protein; and the nucleic acid molecule C is capable of expressing the TaCIPK14-4D protein;
the nucleic acid molecule A is selected from the group consisting of
(a1) A DNA molecule set forth in SEQ ID NO: 1;
(a2) A DNA molecule hybridized with the DNA molecule defined in (a1) under stringent conditions and encoding the TaCIPK14-4A protein; and
(a3) A DNA molecule having at least 99%, at least 95%, at least 90%, at least 85%, or at least 80% identity to any of the DNA sequences defined in (a1)-(a2) and encoding the TaCIPK14-4A protein;
the nucleic acid molecule B is selected from the group consisting of
(b1) A DNA molecule set forth in SEQ ID NO: 2;
(b2) A DNA molecule hybridized with the DNA molecule defined in (b1) under stringent conditions and encoding the TaCIPK14-4B protein; and
(b3) A DNA molecule having at least 99%, at least 95%, at least 90%, at least 85%, or at least 80% homology to any of the DNA sequences defined in (b1)-(b2) and encoding the TaCIPK14-4B protein; and
the nucleic acid molecule C is selected from the group consisting of
(c1) A DNA molecule set forth in SEQ ID NO: 3;
(c2) A DNA molecule hybridized with the DNA molecule defined in (c1) under stringent conditions and encoding the TaCIPK14-4D protein; and
(c3) A DNA molecule having at least 99%, at least 95%, at least 90%, at least 85%, or at least 80% homology to any of the DNA sequences in (c1)-(c2) and encoding the TaCIPK14-4D protein.

22. The method of claim 15, wherein inhibiting expression of a nucleic acid molecule capable of expressing TaCIPK14 protein in a recipient plant is achieved by introducing an RNA interference fragment, an RNA interference vector, a homologous recombinant fragment, a homologous recombinant vector, or a gene editing tool into the recipient plant.

23. The method of claim 15, wherein the gene editing tool is a CRISPR/Cas9 system, wherein a target sequence of the gRNA comprises SEQ ID NO: 7 and/or SEQ ID NO: 8.
